(19) United States
(12) Patent Application Publication
(10) Pub. No.: US 2011/0105343 A1
(43) Pub. Date: May 5, 2011

(54) Systems Biology Approach Predicts Immunogenicity of Vaccines

(75) Inventors: Bali Puledran, Atlanta, GA (US); Rafi Ahmed, Atlanta, GA (US); Eva Lee, Atlanta, GA (US); Troy Querec, Decatur, GA (US)

(73) Assignee: EMORY UNIVERSITY, Atlanta, GA (US)

(21) Appl. No.: 12/935,347

(22) PCT Filed: Nov. 23, 2009
(86) PCT No.: PCT/US09/65563

§ 371 Date: Dec. 20, 2010

Related U.S. Application Data
(60) Provisional application No. 61/116,877, filed on Nov. 21, 2008.

(51) Int. Cl. C12Q 1/02; C12Q 1/68; C40B 30/00

(52) U.S. Cl. 506/7; 435/29; 435/6

(57) ABSTRACT

A major challenge in vaccinology is to prospectively determine vaccine efficacy. Disclosed herein are methods and compositions for identifying early expression “signatures” that predicted immune responses in humans vaccinated with a vaccine.

This application claims the benefit of U.S. Provisional Application No. 61/116,877, filed on Nov. 21, 2009 which is incorporated by reference herein in its entirety.

This application was made with government support under federal grants NIH U19 AI057266, R01 AI048638, R01 DK057665, U54 AI 057157, N01 AI50019, and N01 AI50025. The Government has certain rights in the invention.

### I. BACKGROUND

Millions of people each year receive vaccines that confer immunological protection against viral, bacterial, fungal, and parasitic infections. Yet, despite the historical success of vaccines, little is known about the mechanisms by which vaccines induce these effective immune responses. Moreover, while some vaccines confer long lived immunological protection, other vaccines have short protective lives. What is needed are methods for assessing the efficacy of a vaccine so that the effectiveness in generating an adaptive immune response can be assessed and where appropriate the vaccine can be modified to increase the immunogenicity of the vaccine.

### II. SUMMARY

Disclosed are methods and compositions related to accessing the efficacy of a vaccine. The methods disclosed herein utilize differential expression profiles of genes or proteins and computational analysis to create an expression signature that is predictive of adaptive immune responses. Also disclosed herein are methods of screening subjects for suitability to receive a vaccine.

The methods disclosed herein are broadly applicable to vaccine construction. Thus, disclosed herein are methods of making a vaccine comprising an antigenic element and a regulatory element wherein the regulatory element modifies an expression signature to optimize a desired adaptive immune response.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the cytokine and dendritic cell responses to YF-17D. (a) The maximum fold change in cytokine expression out of days 3 or 7 is calculated and depicted as a heat map with GeneSpring software. (b) Out of the cytokines that are induced by vaccination, IP-10 and IL1A are significantly upregulated on day 7. Data were normalized using the pre-vaccination cytokine level [i.e. Log 2(Cd)−Log 2(C0), where Cd is the cytokine concentration on day d]. (c) The percentage of CD86+ myeloid dendritic cells, plasmacytoid dendritic cells, total monocytes, or inflammatory CD16+ monocytes is first calculated for each day. The Log 2 transformed values for the percentages of CD86+ cells were normalized relative to baseline levels. The change in the percentage of CD86+ positive cells is then calculated for each day relative to day 0 and tested for significance. The determination of significant changes was based on ANOVA followed by Tukey's multiple test comparison on the 15 subjects of Trial 1. * P<0.05, ** P<0.01, *** P<0.001.

FIG. 2 shows the identification of commonly induced genes in two independent vaccine trials. FIG. 2A shows the fold change in expression is calculated for each gene on days 3 and 7 relative to day 0. Genes with Log 2 fold changes >0.5 or <−0.5 in at least 60% of subjects are then selected. The linear expression values for these genes are then analyzed for significance in GeneSpring. FIG. 2B shows that genes with a Benjamini and Hochbery False Discovery Rate less than 0.05 for each trial are then compared. The genes identified as being significantly changed on days 3 or 7 are analyzed level 4 Gene Ontology terms using DAVID to identify associations among the genes. The results are based on 15 subjects in Trial 1 and 10 subjects in Trial 2.

FIG. 3 shows the Genomic signatures of innate immune responses to YF-17D. FIG. 3A shows Ingenuity Pathways Analysis of a subset of genes identified as being regulated significantly (Benjamini and Hochberg false-discovery rate, <0.05) in two independent trials and supplemented with transcription factor binding motif information from TOUCAN for IRF7 and IRF9 (complete network, FIG. 4). FIG. 3B shows a heat map showing kinetics of changes in expression of common genes identified in two independent trials sorted into categories based on DAVID Bioinformatics Database gene descriptions. The heat map colors represent the average expression among the subjects for each time point (given in days at the bottom of each column). FIG. 3c shows changes in relative gene expression have significant correlations between microarray and RT-PCR analysis. Each point represents a single gene at a given time point. FIG. 3D shows analysis of 33 genes identified as being significantly modulated by microarray analysis reveals that 26 genes also have significant modulation as measured by RT-PCR (P<0.05). The heat map represents the gene expression by RT-PCR on days 3 and 7 as a multiple of that on day 0. All genes and time points were first normalized to the average cycling threshold value of expression of the housekeeping genes for 18S ribosomal RNA, ACTB (β-actin) and B2M (β2-microglobulin). The gene expression on days 3 and 7 as a multiple of that on day 0 was then calculated and imported into GeneSpring for heat map production. Data from 1A and 1B are derived from trials 1 and 2, with 15 and 10 subjects, respectively. Data from 1C and 1D are from trial 1, with 15 subjects.

FIG. 4 shows the network of anti-viral genes in response to YF-17D. Ingenuity Pathways Analysis of genes identified in FIG. 3b as being regulated significantly in two independent trials and supplemented with transcription factor binding motif information from TOUCAN for IRF7 and IRF9 (Table 2).

FIG. 5 shows the induction of complement C3a by YF-17D. Plasma concentrations of C3a is measured by ELISA to confirm activation of the complement pathways. The determination of significant changes was based on ANOVA followed by Tukey's multiple test comparison on the 10 subjects of Trial 2. * P<0.05.

FIG. 6 shows that YF-17D induces NF-κB activation via RIG-I and MDA-5. Human embryonic fibroblasts (HEK293 cell line) were cotransfected with plasmids encoding luciferase driven by an NF-κB promoter, plus a plasmid encoding either MDA-5 or RIG-I for 24 hr. Then cells were stimulated with poly-IC or YF-17D for 6 hr or 48 hr. NF-κB induction was detected by luciferase activity. Representative of 2 independent experiments.

FIG. 7 shows the induction of anti-viral genes in PBMCs stimulated in vitro with YF-17D. PBMCs from 2 healthy unvaccinated donors were isolated and plated at 1×106 cells per well in 48-well plates with 1 ml RPMI with 10% FBS and penicillin/streptomycin. The cells were cultured in the presence or absence of YF-17D at a MOI of 1. After 3 and 12 hours, RNA was isolated from the cells and processed for microarray analysis. For these experiments, the Affymetrix Human Genome 133A 2.0 Array was used. This microarray contains a subset of genes found on the Human 133 Plus 2.0 Array, which was used in the analysis of the vaccinees. Genes were selected that were up or down regulated by a factor of 0.5 fold in the Log 2 scale, after either 3 or 12 hours of stimulation with YF-17D, compared to cells cultured in media alone. The student t-test was used to compare YF-17D to media alone at 3 and 12 hours.

FIG. 8 shows variations in the magnitudes of the antigen-specific CD8⁺ T cell and neutralizing antibody responses to YF-17D. FIG. 8A shows Flow cytometry for expression of HLA-DR with CD38, on gated CD3⁺CD8⁺ T cells isolated from blood of YF-17D vaccinees. The red dots and numbers indicate the yellow-fever specific CD8⁺ T cells that stained with the HLA-A2-restricted tetramer (YF-Tet⁺). FIG. 8B shows the correlation between YF-Tet⁺ T cells and HLA-DR⁺CD38⁺CD3⁺CD8⁺ T cells. FIG. 8C shows flow cytometry analysis of granzyme B, CD27, CD28, Bcl-2, Ki67, CD127, CCR5, CD45RA and CCR7 in the blood of YF-17D subjects from trial 1. HLA-DR⁺CD38⁺CD8⁺ T cells (in regions outlined for plots of days 0 and 15) have effector phenotype (red dots) on day 15. FIGS. 8D and 8E show a graph of flow cytometry data comparing day 15 and day 60 CD8⁺ T cell activation and neutralizing antibody titers from 15 subjects in trial 1.

FIG. 9 shows that genomic signatures that correlate with the magnitude of the CD8⁺ T cell response. Genes with a log₂-fold change of >0.5 or <−0.5 in more than 25% of the 15 subjects of trial 1 were first selected, for day 3 versus day 0 and separately for day 7 versus day 0. Next, the slope of the P-value of the percentage of activated CD8⁺ T cells versus the log₂-fold change in gene expression was calculated for each remaining gene. Those genes with P<0.05 were identified as having a significant relationship between early gene expression changes and later CD8⁺ T cell responses. FIG. 9A shows the unsupervised principal component analysis of the gene expression for each subject on both days 3 and 7 revealed that subjects could be segregated on the basis of CD8⁺ T cell responses above and below 3%. FIG. 9B shows a standard correlation cluster analysis in GeneSpring confirmed the segregation of T cell responses into two groups with an approximate cutoff of 3-4% activation.

FIG. 10 shows the Genomic signatures that predict the magnitude of the CD8⁺ T cell responses, using the ClaNC model. The genes identified as having a relationship to the subsequent T cell responses, as described in FIG. 9, were analyzed by ClaNC to develop a predictive model of CD8⁺ T cell responses based on a subset of genes. (a) A process of leave-one-out cross-validation testing the predictive strengths of subsets of genes for ClaNC gene models. (b) The ClaNC gene models developed through cross validation on the first trial of 15 subjects was tested on both trials of 15 and 10 subjects to determine the error rates.

FIG. 11 shows that YF-17D induces eIF2α phosphorylation and stress granule formation. FIG. 11A shows Immunoblot on lysates from human total PBMC or baby hamster kidney cells that were treated with 0.5 mM arsenite for 30 min or YF-17D for the indicated lengths of time. Cell extracts were prepared and probed for eIF2α phosphorylation (top) as well as for total eIF2α abundance (bottom). FIG. 11B shows Fluorescence microscopy of baby hamster kidney cells treated with 0.5 mM arsenite for 30 min or YF-17D (multiplicity of infection 2) overnight before fixing and staining for cytotoxic granule-associated RNA-binding protein-like 1 (TIAR; green). Cells were counterstained with BODIPY 558/568 phalloidin for F-actin (red) and DAPI for nuclei (blue). Scale bars, 5 μm. Results are representative of two independent experiments.

FIG. 12 shows the correlation coefficients and P-values of stress response genes that correlate with the magnitude of the CD8+ T cell response. FIG. 12A shows Calreticulin at Day 3. FIG. 12B shows protein disulfide isomerase family A, member 5 at Day 3. FIG. 12C shows protein disulfide isomerase family A, member 4 at Day 3. FIG. 12d shows protein disulfide isomerase family A, member 4 at Day 7. FIG. 12E shows nuclear receptor subfamily 3, group C, member 1 (glucocorticoid receptor) at Day 3. FIG. 12F shows eukaryotic translation initiation factor 2 alpha kinase 4 at Day 7. The data are from the 15 subjects of Trial 1.

FIG. 13 shows the genomic signatures that correlate with the magnitude of the antibody response. Genes with a Log 2 fold change of >0.5 or <−0.5 in greater than 25% of the subjects are first selected. Next the slope P-value of the day 60 antibody titers versus Log 2 fold change in gene expression was calculated for each remaining gene. Those genes with P<0.05 are identified as having a significant relationship between early gene expression changes and later antibody responses. Unsupervised principle component analysis of the gene expression for each subject on both days 3 and 7 reveals that subjects could be segregated based on antibody titers above and below 170. Data are from the 15 subjects of Trial 1.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Methods of Assessing the Efficacy of a Vaccine

The need to assess the efficacy of a vaccine is tantamount to the ability to insure immunological protection is afforded a subject receiving the vaccine. The determination of efficacy has consequences for vaccine design, dosing regimens, whether a subject needs a booster immunization, the determination of the possible time for which immunological protection can be conferred, the degree of protection, and what individuals will be responsive to a vaccine. Thus disclosed herein are methods for measuring or accessing the efficacy of a vaccine comprising identifying a differential expression signature of a tissue sample from an immunized subject, wherein the presence or absence of one or more innate response elements in expression signature indicates the presence of an adaptive immune response, and wherein the presence of an adaptive immune response indicates an efficacious vaccine.

“Efficacy,” “efficacious,” or “sufficiency” mean the ability to function as intended. For example, an “efficacious” immune response is a response that is able to afford the subject a degree of immune protection from the immunizing antigen. Thus, the present methods disclose methods of assessing the ability of an immune response to provide immune protection against future or current (in the case of a therapeutic vaccine) antigenic encounter. Traditionally, such methods involve antigenic challenge. It is understood that the present methods provide an alternative means to achieve the goal of antigenic challenge while doing so in a predictive or prophetic manner rather than historic. The methods disclosed herein can be used separately or in conjunction with a challenge to determine efficacy or sufficiency.

Immune responses to antigenic encounter are broadly categorized as innate and adaptive immune responses. Innate responses are those responses that are not driven by specificity for a particular antigen, but the presence of the antigen. Innate responses include dendritic cell activation, cytokine and chemokine secretion, and activation of the complement cascade. By contrast, adaptive immune responses, that is, cell-mediated (T cells) and humoral (antibody) responses, are tailored to a particular antigen. Adaptive responses develop after an initial antigenic exposure and form a memory pool of T cells and/or B cells which rapidly respond to an antigen upon subsequent antigen exposure to the same antigen. The adaptive immune responses are structured around the ability to recognize antigenic sequences (primary peptide sequences in the context of MHC for T cells and tertiary sequences for antibodies and B cells). As a consequence variation in a sequence can lead to a lack of adaptive response if the sequence variation is such that cross-reactivity does not occur.

The methods disclosed herein use the expression of innate response elements early in an immune response to be predictive or prophetic markers for the development of an efficacious adaptive immune response. As disclosed herein, “innate response elements” refers to any gene, protein, nucleic acid, or microRNA associated with the expression or regulation of an innate immune response including cytokine and chemokine expression and secretion; dendritic cell activation; and complement cascade. Thus, for example “innate response elements” can include genes or proteins associate with or the regulation of signal transduction, interferon family members, complement, antigen processing, signal transduction, ubiquitination, chemotaxis, cell adhesion, and polymerase activity. Also disclosed herein are methods wherein the innate response element is an innate sensing receptor, a cytoplasmic receptor for oligodenylate synthetases, TNF receptor family members, a transcription factors that regulate type I interferon expression. Examples of innate response elements include but are not limited to OAS1, OAS2, OAS3, RIG-1, EIF2AK2, PKR, IFIH1/MDA-5, TLR7, LGP2, MX1, PLSCR1, RSAD2, TRIM22, TRIMS, GBP1, IFI27, IFI8, IFI44, IFI44L, IFIT1, IFIT2, IFIT3, MX2, PNPT1, RTP4, C3AR1, SERPING1, IRF7, JUN, RGL1, STAT1, CDKN1C, RNF36, FBXO6, HERC5, HERC6, ISG15, UBE2L6, XAF1, CD38, LGALS38P, SIGLEC1, PARP12, PARP9, PARP14, EPSTI1, FAM70A, FER1L3, MS4A4, SAMD9, SAMD9L, TDRD7, CMPK2, DDX60, DDX60L, KLHDC7B, CXCL10, IP-10, MARKS, NEXN, SCL2A6, EIF2AK4, ITGAL/LFA-1, CTBP1, YWHAE, PPP1R144, TLR2, TLR7, TLR8, FAM62B, RGS1, CD69, ALDH3B1, CXCR7, C1QB, ASGR2, ITGAL, MEF2A, BEND4, PFKFB3, TNFRSF17, TPD52, KBTBD7, NAP1L2, and ATP6V1E1.

Thus, for example, disclosed herein are methods wherein the innate response element is a gene associated with the complement system such as C1QB. Also disclosed, for example are methods wherein the innate response element regulates glucose transport and glycolysis, such as SLC2A6; or regulates protein synthesis in response to stress, such as EIF2AK4. It is further understood that the differential expression signature being a relative value can refer to the up or down regulation of a gene or protein expression relative to the control. It is also understood that the expression signature can be correlated to particular arms of the adaptive immune response. For example C1QB, SLC2A6, and EIF2AK4 are associated with T cell responses; whereas TNF receptor superfamily receptor 17 (TNFRSF17) is associated with B cell responses or antibody production.

It is understood that as innate response elements can be used can used in the disclosed methods, genes and proteins associate with the expression and regulation of the adaptive immune response can also be used with the methods herein to assess or measure the efficacy of a vaccine. Example of genes and proteins that can be associated with the expression and regulation of an adaptive immune response can be found in table 5 and include but are not limited to ALDH16A1, ALDH3B1, ASGR2, ATP6V1E1, BIRC3, BNIP3L, BCKDK, CAMKK2, CALR, CRAT, CTSB, CENPB, CXCR6, CXCR7, DEFA4, EMILIN2, ETV3, EIF4G3, FBXO15, GPR18, GBGT1, GAA, GAS2L1, HEATR3, HBA1, HBB, HBZ, HTRA4, FLJ10847, SLC47A1, IMPDH1, MYL4, NANS, NRGN, NAPRT1, NUDT14, PNPLA6, PRAM1, RAB8B, NDRG2, STK17A, SMARCD3, SLC16A5, SLC2A6, SLC25A13, SLC39A11, SAT2, SPON2, TBC1D7, TEP1, THAP11, TCEAL4, TMEM176A, TMOD1, TUFM, TNFSF14, FERMT3, ULK2, WDR40A, ZFP82, ZNF606, ZSWIM5, and ZYX.

Typically immune protection refers to adaptive immune responses. Thus, put another way, the methods disclosed herein provide for determining the efficacy of adaptive immune responses to a vaccine by identifying of differential expression signature of innate response elements, wherein the presence of absence of particular innate response elements indicates the efficacy of the adaptive immune response.

Throughout this application the term “sufficient immune response” is used to describe an immune response of a large enough magnitude to provide an acceptable immune protection to the subject against future antigen encounter. It is understood that immune protection does not necessarily mean prevention of future antigenic encounter (e.g., infection), nor is it limited to a lack of any pathogenic symptoms. “Immune protection” means a prevention of the full onset of a pathogenic condition. Thus in one embodiment a “sufficient immune response” is a response that reduces the symptoms, magnitude, or duration of an infection or other disease condition when compared with an appropriate control. The control can be a subject that is exposed to an antigen before or without a sufficient immune response.

By “effective amount” is meant a therapeutic amount needed to achieve the desired result or results, e.g., establishing an immune response that can confer immunological protection to the subject. It is understood that immunological protection includes but is not limited to prevention of subsequent infections; reduction of the effects or symptoms of subsequent infections or conditions; reduction in the duration of the infection or condition; lessening of severity of a disease or condition; or reduced antigenic load relative to non-treated controls.

It is understood herein that an “immune response” refers to any inflammatory, humoral, or cell-mediated response that occurs for the purpose of eliminating an antigen. Such responses can include, but are not limited to, antibody production, cytokine secretion, complement activity, and cytolytic activity. In one embodiment, the immune response is a antibody response.

The methods disclosed herein describe the use of differential expression signatures to determine efficacy. As used herein, a “differential expression profile” refers to the gene or protein expression pattern following exposure to an antigen. However, a “differential expression signature” refers to a set or pattern of gene or protein that correlates with an adaptive immune response. It is understood, and contemplated herein, that the differential expression signature is the result of performing computational analysis on a genetic or protein expression profile. Therefore, the term “differential expression profile” is not synonymous with “differential expression signature.” Thus, in one aspect, the methods disclosed herein involve identifying a differential expression signature by measuring the differential expression profile of a tissue sample from an immunized subject and identifying innate response elements with significant expression through computational analysis, wherein the innate response elements with significant expression comprise the expression signature of the sample.

Also disclosed herein are methods for identifying, creating, deriving, establishing an expression profile signature of an innate immune response element comprising comparing the expression profile of one or more innate response elements in a tissue sample of an immunized subject to a control sample, wherein the innate response elements with significant differential expression are then correlated with an adaptive immune response using computational analysis; and wherein the innate response elements displaying a correlation to an adaptive immune response comprise the differential expression signature.

The computational analysis can occur by any means known in the art to derive a correlation between an adaptive immune response and an expression profile. Computational analysis algorithms include but are not limited to discriminant analysis via mixed integer programming (DAMIP), Classification to the nearest centroid (ClaNC), GeneSpring's standard correlation with average linkage hierarchical clustering analysis, and RANDOM FOREST®. Thus, for example, disclosed herein are methods for accessing the efficacy of a vaccine comprising identifying a differential expression signature of a tissue sample from an immunized subject, wherein the presence or absence of one or more innate response elements in the expression signature indicates the presence of an adaptive immune response; wherein the presence of an adaptive immune response indicates an efficacious vaccine; wherein the differential expression signature is identified by measuring the differential expression profile of a tissue sample from an immunized subject and identifying innate response elements with significant expression through computational analysis, wherein the innate response elements with significant expression comprise the expression signature of the sample; and wherein the computational analysis is discriminant analysis via mixed integer programming (DAMIP).

The differential expression profile utilized in the methods disclosed herein can be identified by any means known in the art and can relate to the expression of proteins, genes, or microRNAs. For example, an expression profile can be identified through the use Western Blot, RT-PCR, protein array, or gene array.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

Provided that the concentrations are sufficient, the molecular complexes ([Ab-Ag]n) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab-Ag]n), and reagent antigens are used to detect specific antibody ([Ab-Ag]n). If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards that make the procedures more complex. Some of these Group 1 assays date right back to the discovery of antibodies and none of them have an actual “label” (e.g. Ag-enz). Other kinds of immunoassays that are label free depend on immunosensors, and a variety of instruments that can directly detect antibody-antigen interactions are now commercially available. Most depend on generating an evanescent wave on a sensor surface with immobilized ligand, which allows continuous monitoring of binding to the ligand. Immunosensors allow the easy investigation of kinetic interactions and, with the advent of lower-cost specialized instruments, may in the future find wide application in immunoanalysis.

The use of immunoassays to detect a specific protein can involve the separation of the proteins by electophoresis. Electrophoresis is the migration of charged molecules in solution in response to an electric field. Their rate of migration depends on the strength of the field; on the net charge, size and shape of the molecules and also on the ionic strength, viscosity and temperature of the medium in which the molecules are moving. As an analytical tool, electrophoresis is simple, rapid and highly sensitive. It is used analytically to study the properties of a single charged species, and as a separation technique.

Generally the sample is run in a support matrix such as paper, cellulose acetate, starch gel, agarose or polyacrylamide gel. The matrix inhibits convective mixing caused by heating and provides a record of the electrophoretic run: at the end of the run, the matrix can be stained and used for scanning, autoradiography or storage. In addition, the most commonly used support matrices—agarose and polyacrylamide—provide a means of separating molecules by size, in that they are porous gels. A porous gel may act as a sieve by retarding, or in some cases completely obstructing, the movement of large macromolecules while allowing smaller molecules to migrate freely. Because dilute agarose gels are generally more rigid and easy to handle than polyacrylamide of the same concentration, agarose is used to separate larger macromolecules such as nucleic acids, large proteins and protein complexes. Polyacrylamide, which is easy to handle and to make at higher concentrations, is used to separate most proteins and small oligonucleotides that require a small gel pore size for retardation.

Proteins are amphoteric compounds; their net charge therefore is determined by the pH of the medium in which they are suspended. In a solution with a pH above its isoelectric point, a protein has a net negative charge and migrates towards the anode in an electrical field. Below its isoelectric point, the protein is positively charged and migrates towards the cathode. The net charge carried by a protein is in addition independent of its size—i.e., the charge carried per unit mass (or length, given proteins and nucleic acids are linear macromolecules) of molecule differs from protein to protein. At a given pH therefore, and under non-denaturing conditions, the electrophoretic separation of proteins is determined by both size and charge of the molecules.

Sodium dodecyl sulphate (SDS) is an anionic detergent which denatures proteins by “wrapping around” the polypeptide backbone—and SDS binds to proteins fairly specifically in a mass ratio of 1.4:1. In so doing, SDS confers a negative charge to the polypeptide in proportion to its length. Further, it is usually necessary to reduce disulphide bridges in proteins (denature) before they adopt the random-coil configuration necessary for separation by size; this is done with 2-mercaptoethanol or dithiothreitol (DTT). In denaturing SDS-PAGE separations therefore, migration is determined not by intrinsic electrical charge of the polypeptide, but by molecular weight.

Determination of molecular weight is done by SDS-PAGE of proteins of known molecular weight along with the protein to be characterized. A linear relationship exists between the logarithm of the molecular weight of an SDS-denatured polypeptide, or native nucleic acid, and its Rf. The Rf is calculated as the ratio of the distance migrated by the molecule to that migrated by a marker dye-front. A simple way of determining relative molecular weight by electrophoresis (Mr) is to plot a standard curve of distance migrated vs. log 10 MW for known samples, and read off the log Mr of the sample after measuring distance migrated on the same gel.

In two-dimensional electrophoresis, proteins are fractionated first on the basis of one physical property, and, in a second step, on the basis of another. For example, isoelectric focusing can be used for the first dimension, conveniently carried out in a tube gel, and SDS electrophoresis in a slab gel can be used for the second dimension. One example of a procedure is that of O'Farrell, P. H., High Resolution Two-dimensional Electrophoresis of Proteins, J. Biol. Chem. 250:4007-4021 (1975), herein incorporated by reference in its entirety for its teaching regarding two-dimensional electrophoresis methods. Other examples include but are not limited to, those found in Anderson, L and Anderson, N G, High resolution two-dimensional electrophoresis of human plasma proteins, Proc. Natl. Acad. Sci. 74:5421-5425 (1977), Ornstein, L., Disc electrophoresis, L. Ann. N.Y. Acad. Sci. 121:321349 (1964), each of which is herein incorporated by reference in its entirety for teachings regarding electrophoresis methods. Laemmli, U. K., Cleavage of structural proteins during the assembly of the head of bacteriophage T4, Nature 227:680 (1970), which is herein incorporated by reference in its entirety for teachings regarding electrophoresis methods, discloses a discontinuous system for resolving proteins denatured with SDS. The leading ion in the Laemmli buffer system is chloride, and the trailing ion is glycine. Accordingly, the resolving gel and the stacking gel are made up in Tris-HCl buffers (of different concentration and pH), while the tank buffer is Tris-glycine. All buffers contain 0.1% SDS.

One example of an protein expression profile assay as contemplated in the current methods is Western blot analysis. Western blotting or immunoblotting allows the determination of the molecular mass of a protein and the measurement of relative amounts of the protein present in different samples. Detection methods include chemiluminescence and chromagenic detection. Standard methods for Western blot analysis can be found in, for example, D. M. Bollag et al., Protein Methods (2d edition 1996) and E. Harlow & D. Lane, Antibodies, a Laboratory Manual (1988), U.S. Pat. No. 4,452,901, each of which is herein incorporated by reference in their entirety for teachings regarding Western blot methods. Generally, proteins are separated by gel electrophoresis, usually SDS-PAGE. The proteins are transferred to a sheet of special blotting paper, e.g., nitrocellulose, though other types of paper, or membranes, can be used. The proteins retain the same pattern of separation they had on the gel. The blot is incubated with a generic protein (such as milk proteins) to bind to any remaining sticky places on the nitrocellulose. An antibody is then added to the solution which is able to bind to its specific protein.

The attachment of specific antibodies to specific immobilized antigens can be readily visualized by indirect enzyme immunoassay techniques, usually using a chromogenic substrate (e.g. alkaline phosphatase or horseradish peroxidase) or chemiluminescent substrates. Other possibilities for probing include the use of fluorescent or radioisotope labels (e.g., fluorescein, ¹²⁵I). Probes for the detection of antibody binding can be conjugated anti-immunoglobulins, conjugated staphylococcal Protein A (binds IgG), or probes to biotinylated primary antibodies (e.g., conjugated avidin/streptavidin).

The power of the technique lies in the simultaneous detection of a specific protein by means of its antigenicity, and its molecular mass. Proteins are first separated by mass in the SDS-PAGE, then specifically detected in the immunoassay step. Thus, protein standards (ladders) can be run simultaneously in order to approximate molecular mass of the protein of interest in a heterogeneous sample.

The gel shift assay or electrophoretic mobility shift assay (EMSA) can be used to detect the interactions between DNA binding proteins and their cognate DNA recognition sequences, in both a qualitative and quantitative manner. Exemplary techniques are described in Ornstein L., Disc electrophoresis—I: Background and theory, Ann. NY Acad. Sci. 121:321-349 (1964), and Matsudiara, P T and D R Burgess, S D S microslab linear gradient polyacrylamide gel electrophoresis, Anal. Biochem. 87:386-396 (1987), each of which is herein incorporated by reference in its entirety for teachings regarding gel-shift assays.

In a general gel-shift assay, purified proteins or crude cell extracts can be incubated with a labeled (e.g., ³²P-radiolabeled) DNA or RNA probe, followed by separation of the complexes from the free probe through a nondenaturing polyacrylamide gel. The complexes migrate more slowly through the gel than unbound probe. Depending on the activity of the binding protein, a labeled probe can be either double-stranded or single-stranded. For the detection of DNA binding proteins such as transcription factors, either purified or partially purified proteins, or nuclear cell extracts can be used. For detection of RNA binding proteins, either purified or partially purified proteins, or nuclear or cytoplasmic cell extracts can be used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions. Refer to Promega, Gel Shift Assay FAQ, available at <http://www.promega.com/faq/gelshfaq.html> (last visited Mar. 25, 2005), which is herein incorporated by reference in its entirety for teachings regarding gel shift methods.

Gel shift methods can include using, for example, colloidal forms of COOMASSIE (Imperial Chemicals Industries, Ltd) blue stain to detect proteins in gels such as polyacrylamide electrophoresis gels. Such methods are described, for example, in Neuhoff et al., Electrophoresis 6:427-448 (1985), and Neuhoff et al., Electrophoresis 9:255-262 (1988), each of which is herein incorporated by reference in its entirety for teachings regarding gel shift methods. In addition to the conventional protein assay methods referenced above, a combination cleaning and protein staining composition is described in U.S. Pat. No. 5,424,000, herein incorporated by reference in its entirety for its teaching regarding gel shift methods. The solutions can include phosphoric, sulfuric, and nitric acids, and Acid Violet dye.

Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g. where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, N.J.) and specialized chip designs, such as engineered microchannels in a plate (e.g., The Living Chip™, Biotrove, Woburn, Mass.) and tiny 3D posts on a silicon surface (Zyomyx, Hayward Calif.). Particles in suspension can also be used as the basis of arrays, providing they are coded for identification; systems include color coding for microbeads (Luminex, Austin, Tex.; Bio-Rad Laboratories) and semiconductor nanocrystals (e.g., QDots™, Quantum Dot, Hayward, Calif.), and barcoding for beads (UltraPlex™, SmartBead Technologies Ltd, Babraham, Cambridge, UK) and multimetal microrods (e.g., Nanobarcodes™ particles, Nanoplex Technologies, Mountain View, Calif.). Beads can also be assembled into planar arrays on semiconductor chips (LEAPS technology, BioArray Solutions, Warren, N.J.).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system (Prolinx, Bothell, Wash.) reversible covalent coupling is achieved by interaction between the protein derivatised with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, Mass.), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilised on a surface such as titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g. Packard Biosciences] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85 sq microns, equivalent to 25 million spots per sq cm, at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, Ariz.), rolling circle DNA amplification (Molecular Staging, New Haven Conn.), mass spectrometry (Intrinsic Bioprobes; Ciphergen, Fremont, Calif.), resonance light scattering (Genicon Sciences, San Diego, Calif.) and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

Antibody arrays have the required properties of specificity and acceptable background, and some are available commercially (BD Biosciences, San Jose, Calif.; Clontech, Mountain View, Calif.; BioRad; Sigma, St. Louis, Mo.). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; BioInvent, Lund, Sweden; Affitech, Walnut Creek, Calif.; Biosite, San Diego, Calif.). In addition to the conventional antibodies, Fab and scFv fragments, single V-domains from camelids or engineered human equivalents (Domantis, Waltham, Mass.) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A (Affibody, Bromma, Sweden), ‘Trinectins’ based on fibronectins (Phylos, Lexington, Mass.) and ‘Anticalins’ based on the lipocalin structure (Pieris Proteolab, Freising-Weihenstephan, Germany). These can be used on capture arrays in a similar fashion to antibodies and may have advantages of robustness and ease of production.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, Calif.).

Another methodology which can be used diagnostically and in expression profiling is the ProteinChip® array (Ciphergen, Fremont, Calif.), in which solid phase chromatographic surfaces bind proteins with similar characteristics of charge or hydrophobicity from mixtures such as plasma or tumour extracts, and SELDI-TOF mass spectrometry is used to detection the retained proteins.

Large-scale functional chips have been constructed by immobilizing large numbers of purified proteins and used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g. via a His tag, and immobilized. Cell free protein transcription/translation is a viable alternative for synthesis of proteins which do not express well in bacterial or other in vivo systems.

For detecting protein-protein interactions, protein arrays can be in vitro alternatives to the cell-based yeast two-hybrid system and may be useful where the latter is deficient, such as interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilised on a microarray. Large-scale ‘proteome chips’ promise to be very useful in identification of functional interactions, drug screening, etc. (Proteometrix, Branford, Conn.).

As a two-dimensional display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

A multiplexed bead assay, such as, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

The methods disclosed herein comprise assessing/measuring the efficacy or sufficiency of an immune response to a selected antigen in a subject. The disclosed methods utilize tissue samples from the subject to provide the basis for assessment. Such tissue samples can include, but are not limited to, blood (including peripheral blood and peripheral blood mononuclear cells), tissue biopsy samples (e.g., spleen, liver, bone marrow, thymus, lung, kidney, brain, salivary glands, skin, lymph nodes, and intestinal tract), and specimens acquired by pulmonary lavage (e.g., bronchoalveolar lavage (BAL)). Thus it is understood that the tissue sample can be from both lymphoid and non-lymphoid tissue. Examples of non-lymphoid tissue include but are not limited to lung, liver, kidney, and gut. Lymphoid tissue includes both primary and secondary lymphoid organs such as the spleen, bone marrow, thymus, and lymph nodes.

The methods disclosed herein relate to assessing efficacy of a vaccine. It is understood and herein contemplated that a vaccine refers to any composition designed to elicit a prophylactic or therapeutic adaptive immune response against an antigen. The vaccine can comprise a live attenuated or killed pathogen or the vaccine can be a subunit vaccine comprising a portion of a larger antigen, for example, a protein, peptide, DNA, or toxoid. Furthermore, the vaccine can comprise pharmaceutically acceptable carrier.

It is understood and contemplated herein that the vaccines assessed or measured using the methods disclosed herein can be administered to a subject via any means known in the art and appropriate given the type of vaccine or vaccine antigen. Specifically contemplated are vaccines delivered by mist, injection, sublingual immunotherapy (SLIT), gene gun, and patch or lotion. Injections can be subcutaneous, intradermal, intramuscular, intravenous, and intraplureal. Nucleic acids such as DNA, or RNA and peptide vaccines can be delivered naked or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.). Vectors can be viral vectors such as retroviral vectors, adenoviral vectors, adeno-associated viral vectors, or any other viral vector known in the art including foamy viral vectors.

As one example, vector delivery can be via a viral system, such as a retroviral vector system which can package a recombinant retroviral genome

It is also understood that the antigen against which an immune response is elicited can be of viral, bacterial, fungal, parasitic, or cancerous origin.

Viral antigens can include any peptide, polypeptide, or protein from a virus. The viral antigen can be from an RNA or DNA virus. The RNA or DNA virus can be positive sense single stranded RNA virus (positive sense ssRNA), a negative-sense single stranded RNA virus (negative sense ssRNA), a double stranded RNA virus (dsRNA), a single stranded DNA virus (ssDNA), or a double stranded DNA virus (dsDNA). Thus, in one embodiment the antigen can be an antigen from a virus of the viral families including but not limited to Coronaviridae, Flaviviridae, Picornaviridae, Togaviridae, Filoviridae, Paramyxoviridae, Orthomyxoviridae, Rhabdoviridae, Bunyaviridae, Reoviridae, Herpesviridae, Adenoviridae, Pappilomaviridae, and Poxyiridae. In another embodiment the antigen can be an antigen from a virus selected from the group consisting of Herpes Simplex virus-1, Herpes Simplex virus-2, Varicella-Zoster virus, Epstein-Barr virus, Cytomegalovirus, Human Herpes virus-6, Human Herpes virus-7, Human Herpes virus-8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

Also disclosed are methods wherein the antigen is a bacterial antigen. The bacterial antigen can be from a gram positive or gram negative bacteria. The antigen, for example, can be a peptide, polypeptide, or protein selected from the group of bacteria consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pneumoniae, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

Also disclosed are methods wherein the antigen is a fungal antigen. The antigen can be, for example, a peptide, polypeptide, or protein selected from the group of fungi consisting of Candida albicans, Cryptococcus neoformans, Histoplama capsulatum, Aspergillus fumigatus, Coccidiodes immitis, Paracoccidiodes brasiliensis, Blastomyces dermitidis, Pneomocystis carnii, Penicillium marneffi, and Alternaria alternata.

Also disclosed are methods wherein the antigen is a parasite antigen. The antigen can be, for example, a peptide, polypeptide, or protein selected from the group of parasitic organisms consisting of Toxoplasma gondii, Plasmodium falciparum, Plasmodium vivax, Plasmodium malariae, other Plasmodium species, Trypanosoma brucei, Trypanosoma cruzi, Leishmania major, other Leishmania species, Schistosoma mansoni, other Schistosoma species, and Entamoeba histolytica.

Also disclosed are methods wherein the antigen is a toxin. It is understood that such toxins can include but are not limited to Abrin, Conotoxins Diacetoxyscirpenol Bovine spongiform encephalopathy agent, Ricin, Saxitoxin, Tetrodotoxin, epsilon toxin, Botulinum neurotoxins, Shigatoxin, Staphylococcal enterotoxins, T-2 toxin, Diphtheria toxin, Tetanus toxoid, and pertussis toxin.

Also disclosed are methods wherein the antigen is a cancer-related antigen. The antigen can be, for example, a peptide, polypeptide, or protein selected from the group of cancers consisting of lymphomas (Hodgkins and non-Hodgkins), B cell lymphoma, T cell lymphoma, myeloid leukemia, leukemias, mycosis fungoides, carcinomas, carcinomas of solid tissues, squamous cell carcinomas, adenocarcinomas, sarcomas, gliomas, blastomas, neuroblastomas, plasmacytomas, histiocytomas, melanomas, adenomas, hypoxic tumors, myelomas, AIDS-related lymphomas or sarcomas, metastatic cancers, bladder cancer, brain cancer, nervous system cancer, squamous cell carcinoma of head and neck, neuroblastoma/glioblastoma, ovarian cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, hematopoietic cancers, testicular cancer, colo-rectal cancers, prostatic cancer, or pancreatic cancer.

The present methods can also be used to the efficacy of immune responses to an antigen related to an autoimmune or inflammatory condition. Such conditions include but are not limited to asthma, rheumatoid arthritis, reactive arthritis, spondylarthritis, systemic vasculitis, insulin dependent diabetes mellitus, multiple sclerosis, experimental allergic encephalomyelitis, Sjögren's syndrome, graft versus host disease, inflammatory bowel disease including Crohn's disease, ulcerative colitis, ischemia reperfusion injury, myocardial infarction, Alzheimer's disease, transplant rejection (allogeneic and xenogeneic), thermal trauma, any immune complex-induced inflammation, glomerulonephritis, myasthenia gravis, cerebral lupus, Guillaine-Barre syndrome, vasculitis, systemic sclerosis, anaphylaxis, catheter reactions, atheroma, infertility, thyroiditis, ARDS, post-bypass syndrome, hemodialysis, juvenile rheumatoid, Behcets syndrome, hemolytic anemia, pemphigus, bulbous pemphigoid, stroke, atherosclerosis, and scleroderma. In particular, the antigen can comprise an amyloid antigen (e.g., amyloid β peptide) thus providing an assessment of an immune response to Alzheimer's disease. Thus, disclosed herein are methods of assessing the effectiveness of a therapy for an autoimmune disease comprising obtaining peripheral blood mononuclear cells (PBMC) from the subject and measuring the presence of antibody secreting cells (ASC) in the PBMC, wherein the absence of ASC indicates an effective therapy.

It is contemplated herein that differential expression signatures can be identified/created to encompass an antigen specific signatures or more universal differential expression signatures. For example, a differential expression signature may be identified that is specific only to the YF-17D vaccine strain of Yellow Fever. Alternatively, an expression signature may be identified/created that encompasses all Yellow Fever viruses. Similarly, more universal differential expression signatures can be devised that cover all members of a viral family or bacterial genus. Moreover, differential expression signatures may be identified that encompass broader classifications of pathogens such as positive-sense single stranded RNA virus (e.g., Coronaviridae, Flaviviridae, Picornaviridae, and Togaviridae), negative-sense single stranded RNA virus (e.g., Filoviridae, Paramyxoviridae, Orthomyxoviridae, Rhabdoviridae, and Bunyaviridae), double stranded RNA viruses (e.g., Reoviridae), single stranded DNA viruses, double stranded DNA viruses (e.g., Herpesviridae, Adenoviridae, Pappilomaviridae, and Poxyiridae), all RNA or DNA viruses, gram positive bacteria, or gram negative bacteria. Thus, contemplated herein are differential expression signatures that are common to all viral family members of Coronaviridae, Flaviviridae, Picornaviridae, Togaviridae, Filoviridae, Paramyxoviridae, Orthomyxoviridae, Rhabdoviridae, Bunyaviridae, Reoviridae, Herpesviridae, Adenoviridae, Pappilomaviridae, or Poxyiridae. Also disclosed are differential expression signatures that are common to all.

Such approaches are of broad value in vaccinology in at least two different ways. First, the identification of molecular signatures of vaccine efficacy could have a public health use in identifying vaccinees who are unlikely to respond well to a vaccine, or in identifying individuals with sub-optimal responses among high risk populations, such as infants or the elderly. In this context, whether the signatures identified with YF-17D can also predict the immunogenicity of other vaccines remains to be determined. It is contemplated herein that a universal “archetypal” signature that predicts the T cell immunogenicity of all vaccines, and another archetypal signature that predicts the B cell immunogenicity of all vaccines can be developed using the methods disclosed herein. At the other extreme, also disclosed are methods in which each vaccine had a unique signature. Thus, one could imagine a cluster of signatures (“meta signatures”) or correlates that predict various aspects of T cell immunogenicity. Similarly for the humoral response, those vaccines that stimulate long-lived plasma cells producing high-affinity antibodies would share a common innate immune signature. Other vaccines that relied on opsonization antibodies for protection, such as the meningococcal or pneumococcal vaccines, will have a different innate immune signature, and so on. Thus a cluster of correlates (or meta signatures), predict various aspects of B cell immunogenicity. Similarly, a different cluster of correlates could exist that predict protective immunity that is not mediated by T or B cell-dependent mechanisms, but involves other mechanisms mediated perhaps by natural killer cells or the activation of stress responses or reactive oxygen species. The elucidation of such meta signatures facilitates not only the rapid screening of vaccines, but also the stimulation of new hypotheses on how vaccines mediate protective immune responses. The realization of these challenges could ultimately lead to the development of a “Vaccine Chip,” which would consist of a few hundred gene probe sets, that can identify predictive signatures for all of the correlates of immunogenicity and protection.

It is contemplated herein that the same information that is used to assess or measure efficacy of a vaccine can be used to improve the design of a vaccine. For example, once a differential expression signature has been created/identified, if necessary, given the expression signature of the vaccine and the desired adaptive immune response, a vaccine can be modified to enhance or suppress expression of one or more innate response elements. Therefore, disclosed herein are vaccines comprising one or more immunogenic elements which stimulate an adaptive immune response and one or more regulatory elements to stimulate or inhibit expression of one or more innate response elements. Also disclosed are methods of increasing the efficacy of a vaccine comprising modifying the vaccine to stimulate or inhibit one or more innate response elements.

The disclosed methods can be used as a component in a larger system to provide as an output the efficacy of a vaccine. Thus, for example, a system can comprise a means for identifying a differential expression profile such as a gene or protein array chip, a reader for the array chip, software for the generation and interpretation of the expression levels of genes or protein in the array, software to further run computational analysis on the expression profiles of genes or proteins on the array and correlate the results with an adaptive immune response, and a processor such as a computer to run the array reader and the software programs. Thus, in one aspect, disclosed herein are systems for determining a differential expression signature comprising a computer, a differential expression array, and software which takes the measurements from the array and applies the expression profile results to a computational analysis algorithm.

### C. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

The yellow fever vaccine (YF-17D) is one of the most effective vaccines ever made; in the past 65 years, it has been administered to over 600 million people globally. YF-17D was developed empirically in the 1930s by Max Theiler, who attenuated the pathogenic Asibi strain of yellow fever virus. A single injection of YF-17D induces a broad spectrum of immune responses, including cytotoxic T lymphocytes (CTLs), a mixed T helper type I (TH1)-TH2 profile, and neutralizing antibodies that can persist for up to 30 years. The mechanism of protection is thought to be mediated by neutralizing antibodies, although cytotoxic T cells also likely to be important.

Because of its longstanding use and efficacy, YF-17D was used as a model to understand the early immune mechanisms-frequently termed the ‘innate response’-underlying this efficacy was believed to be of value in designing new vaccines against other infections. Recent advances have demonstrated a fundamental role for the innate immune system, particularly Toll-like receptors (TLRs) and antigen-presenting cells such as dendritic cells (DCs), in controlling adaptive immune responses. Consistent with this, it was recently shown that YF-17D infects DCs and signals through multiple TLRs on distinct subsets of these DCs. Such immunological ‘deconstruction’ of the mechanisms responsible for the efficacy of an established model vaccine such as YF-17D provide insights into the design of new vaccines against emerging infections and global pandemics.

Disclosed herein, a multivariate analysis of the innate immune responses in humans after vaccination with YF-17D was performed to identify innate immune signatures that are sufficient to predict the subsequent adaptive immune response. To do this, high-throughput technologies, such as gene expression profiling, multiplex analysis of cytokines and chemokines, and multiparameter flow cytometry, were used combined with computational modeling.

a) Results

(1) YF-17D Vaccination Induces a Network of Antiviral Genes

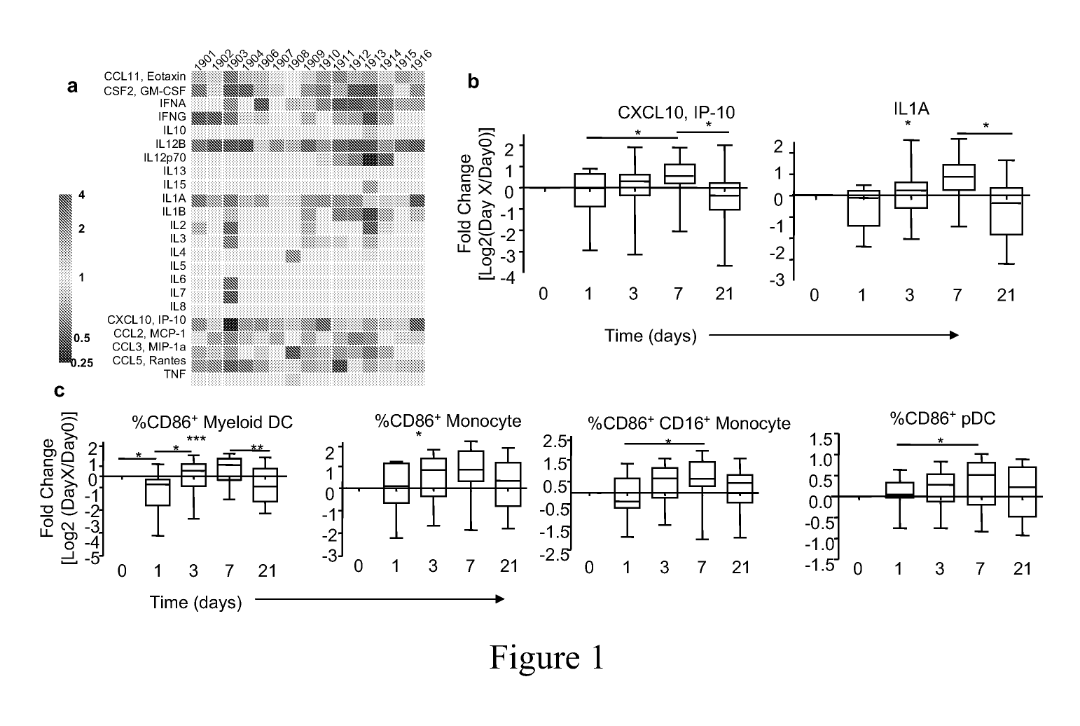

Fifteen healthy humans who had not been previously vaccinated with YF-17D were vaccinated and acquired blood samples at various time points. First, the protein cytokine response was studied in the blood of vaccinees at days 0, 1, 3, 7 and 21 after vaccination, using a 24-plex Luminex assay. Only the chemokine IP-10 (CXCL10, A003787) and the cytokine interleukin 1α (IL-1α) were significantly induced at any given time point, relative to their expression on day 0 (P<0.05; FIG. 1a, and 1b). Next the frequency and activation status of antigen-presenting cells, including DCs and monocytes, was evaluated in the blood at various times after vaccination. There were increases in the percentages of CD86+ myeloid DCs, plasmacytoid DCs, monocytes and CD14+CD16+ inflammatory monocytes at day 7 after vaccination, compared to that on day 0 or 1 (FIG. 1c).

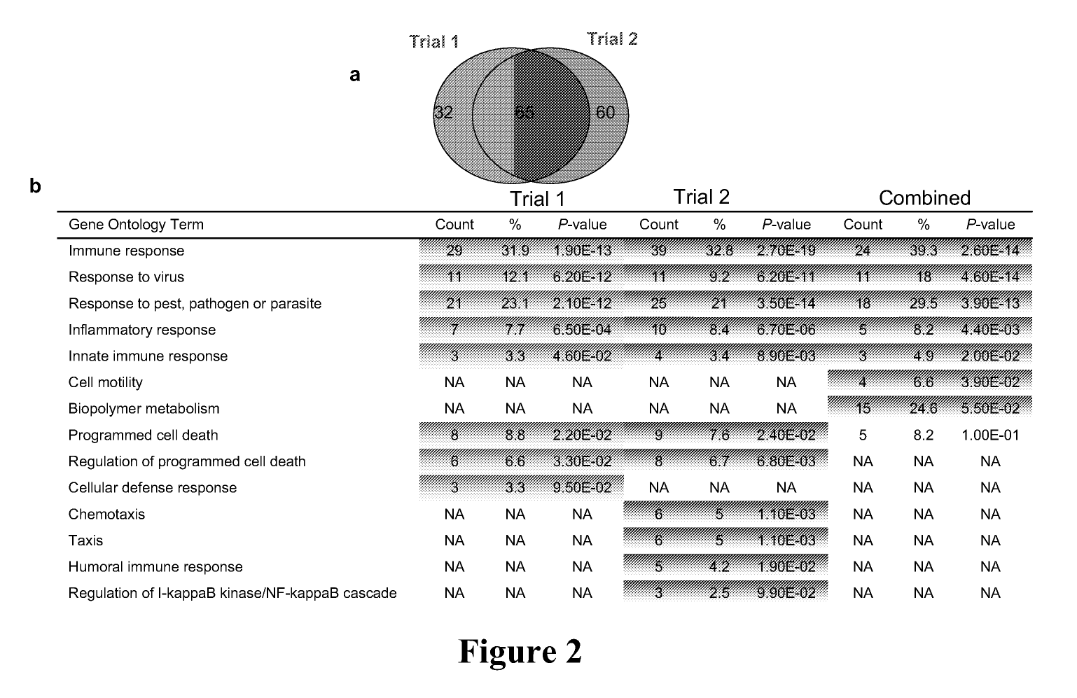

To gain a global perspective of the innate response to YF-17D, transcriptional profiling of total peripheral blood mononuclear cells (PBMCs) from the 15 subjects (trial 1) was performed. For this analysis, the Affymetrix Human Genome U133 Plus 2.0 Array was used. The baseline normalized log 2 gene expression values were first filtered on the basis of the criterion that >60% of the subjects either upregulated or downregulated those genes by at least a factor of ±0.5 on days 3 or 7. The differential expression of these genes over time was analyzed for statistical significance by one-way analysis of variance (ANOVA); P-values were calculated for each gene over the time course of days 0, 1, 3, 7 and 21 by combining the data for all the subjects. The calculations were performed on the log 2-fold change in gene expression for day d versus day 0. To limit the detection of false positives, the P-values were adjusted by the Benjamini and Hochberg false-discovery-rate method with a cutoff of 0.05. This resulted in a list of 97 genes modulated by YF-17D vaccination (FIG. 2a). To confirm these results, a similar analysis was performed in an independent second trial of ten subjects who were vaccinated 1 year later with YF-17D. From this second trial (trial 2), a list of 125 YF-17D-modulated genes was identified, of which 65 were also identified in the initial trial (FIG. 2a). Analyzing the dataset by an independent method, an ANOVA was run on the entire dataset without any prefiltering. Twenty-two genes were obtained, which were a subset of the 65 genes identified using the first strategy (Table 1 and Methods, which includes a detailed discussion of both methods). However, this second method excluded many genes that could be independently verified by RT-PCR or even at the protein level (Table 1).

TABLE 1
Strategies used to identify genes induced by YF-17D in the majority of vaccinees.

| | | Select | ANOVA | Pre- | | RT-PCR | ANOVA Only | Pre-Filtered |
|---|---|---|---|---|---|---|---|---|
| Gene ID | Symbol | Aliases | Only | Filtered | RT-PCR | P-value | vs RT-PCR | vs RT-PCR |
| Hs.118633 | OASL | | 1 | 1 | 1 | 0.00048 | Confirmed | Confirmed |
| Hs.12646 | PARP12 | | 0 | 1 | 1 | 0.00656 | Not Detected but | Confirmed |
| | | | | | | | Confirmed | |
| Hs.130759 | PLSCR1 | | 0 | 1 | 1 | 0.00056 | Not Detected but | Confirmed |
| | | | | | | | Confirmed | |
| Hs.131431 | EIF2AK2 | PKR | 1 | 1 | 1 | 0.00377 | Confirmed | Confirmed |
| Hs.137007 | KLHDC7B | | 1 | 1 | 0 | NA | Not Tested | Not Tested |
| Hs.163173 | IFIH1 | MDA-5 | 0 | 1 | 1 | 0.00218 | Not Detected but | Confirmed |
| | | | | | | | Confirmed | |
| Hs.166120 | IRF7 | | 1 | 1 | 1 | 0.00010 | Confirmed | Confirmed |
| Hs.17518 | RSAD2 | | 1 | 1 | 1 | 0.00064 | Confirmed | Confirmed |
| Hs.190622 | DDX58 | RIG-I | 0 | 1 | 1 | 0.01599 | Not Detected but | Confirmed |
| | | | | | | | Confirmed | |
| Hs.193842 | TDRD7 | | 0 | 1 | 0 | NA | 0 | Not Tested |
| Hs.20315 | IFIT1 | | 0 | 1 | 0 | NA | 0 | Not Tested |
| Hs.26663 | HERC5 | | 1 | 1 | 1 | 0.00016 | Confirmed | Confirmed |
| Hs.31869 | SIGLEC1 | | 1 | 1 | 1 | 0.00005 | Confirmed | Confirmed |
| Hs.325960 | MS4A4 | | 0 | 1 | 0 | NA | 0 | Not Tested |
| Hs.370515 | TRIM5 | | 0 | 1 | 0 | NA | 0 | Not Tested |
| Hs.384598 | SERPING1 | C1IN | 0 | 1 | 1 | 0.00838 | Not Detected but | Confirmed |
| | | | | | | | Confirmed | |
| Hs.388733 | PNPT1 | | 0 | 1 | 0 | NA | 0 | Not Tested |
| Hs.389724 | IFI44L | | 1 | 1 | 1 | 0.00000 | Confirmed | Confirmed |
| Hs.414332 | OAS2 | | 1 | 1 | 1 | 0.00575 | Confirmed | Confirmed |
| Hs.425777 | UBE2L6 | | 0 | 1 | 0 | NA | 0 | Not Tested |
| Hs.43388 | RTP4 | | 0 | 1 | 0 | NA | 0 | Not Tested |
| Hs.437563 | FAM70A | | 1 | 1 | 1 | 0.41473 | Not Confirmed | Not Confirmed |
| Hs.437609 | IFIT2 | | 0 | 1 | 0 | NA | 0 | Not Tested |
| Hs.441975 | XAF1 | | 0 | 1 | 1 | 0.00005 | Not Detected but | Confirmed |
| | | | | | | | Confirmed | |
| Hs.443036 | TLR7 | | 0 | 1 | 1 | 0.04090 | Not Detected but | Confirmed |
| | | | | | | | Confirmed | |
| Hs.458485 | ISG15 | | 1 | 1 | 1 | 0.00005 | Confirmed | Confirmed |
| Hs.464419 | FBXO6 | | 0 | 1 | 0 | NA | 0 | Not Tested |
| Hs.47338 | IFIT3 | | 0 | 1 | 0 | NA | 0 | Not Tested |
| Hs.479214 | CD38 | | 0 | 1 | 1 | 0.03109 | Not Detected but | Confirmed |
| | | | | | | | Confirmed | |
| Hs.489118 | SAMD9L | | 0 | 1 | 0 | NA | 0 | Not Tested |
| Hs.489254 | RNF36 | | 0 | 1 | 0 | NA | 0 | Not Tested |
| Hs.497148 | RGL1 | | 1 | 1 | 0 | NA | Not Tested | Not Tested |
| Hs.500572 | FER1L3 | | 0 | 1 | 0 | NA | 0 | Not Tested |
| Hs.501778 | TRIM22 | | 0 | 1 | 0 | NA | 0 | Not Tested |
| Hs.514535 | LGALS3BP | | 1 | 1 | 0 | NA | Not Tested | Not Tested |
| Hs.517307 | MX1 | | 1 | 1 | 1 | 0.00005 | Confirmed | Confirmed |
| Hs.518200 | PARP9 | | 0 | 1 | 1 | 0.00117 | Not Detected but | Confirmed |
| | | | | | | | Confirmed | |
| Hs.518203 | PARP14 | | 0 | 1 | 1 | 0.09838 | 0 | Not Confirmed |
| Hs.518448 | LAMP3 | | 1 | 1 | 1 | 0.37645 | Not Confirmed | Not Confirmed |
| Hs.519909 | MARCKS | | 0 | 1 | 0 | NA | 0 | Not Tested |
| Hs.523847 | IFI6 | | 1 | 1 | 0 | NA | Not Tested | Not Tested |
| Hs.524760 | OAS1 | | 0 | 1 | 1 | 0.00116 | Not Detected but | Confirmed |
| | | | | | | | Confirmed | |
| Hs.525704 | JUN | | 0 | 1 | 1 | 0.00007 | Not Detected but | Confirmed |
| | | | | | | | Confirmed | |
| Hs.528634 | OAS3 | | 0 | 1 | 1 | 0.00188 | Not Detected but | Confirmed |
| | | | | | | | Confirmed | |
| Hs.529317 | HERC6 | | 1 | 1 | 0 | NA | Not Tested | Not Tested |
| Hs.532634 | IFI27 | | 1 | 1 | 0 | NA | Not Tested | Not Tested |
| Hs.535011 | DDX60L | | 0 | 1 | 0 | NA | 0 | Not Tested |
| Hs.546467 | EPSTI1 | | 1 | 1 | 1 | 0.00070 | Confirmed | Confirmed |
| Hs.55918 | DHX58 | LGP2 | 1 | 1 | 1 | 0.04016 | Confirmed | Confirmed |
| Hs.591148 | C3AR1 | | 0 | 1 | 1 | 0.07323 | 0 | Not Confirmed |
| Hs.591710 | DDX60 | | 0 | 1 | 0 | NA | 0 | Not Tested |
| Hs.598628 | N/A | | 1 | 1 | 0 | NA | Not Tested | Not Tested |
| Hs.604233 | CDKN1C | | 0 | 1 | 0 | NA | 0 | Not Tested |
| Hs.604861 | N/A | | 0 | 1 | 0 | NA | 0 | Not Tested |
| Hs.62661 | GBP1 | | 0 | 1 | 1 | 0.07072 | 0 | Not Confirmed |
| Hs.632387 | NEXN | | 0 | 1 | 0 | NA | 0 | Not Tested |
| Hs.632586 | CXCL10 | IP10 | 0 | 1 | 1 | 0.22878 | 0 | Not Confirmed |
| Hs.633719 | N/A | | 0 | 1 | 0 | NA | 0 | Not Tested |
| Hs.642990 | STAT1 | | 0 | 1 | 0 | NA | 0 | Not Tested |
| Hs.648696 | N/A | | 0 | 1 | 0 | NA | 0 | Not Tested |
| Hs.651258 | N/A | | 0 | 1 | 1 | 0.05277 | 0 | Not Confirmed |
| Hs.65641 | SAMD9 | | 0 | 1 | 0 | NA | 0 | Not Tested |
| Hs.7155 | CMPK2 | | 0 | 1 | 0 | NA | 0 | Not Tested |
| Hs.82316 | IFI44 | | 1 | 1 | 1 | 0.00000 | Confirmed | Confirmed |
| Hs.926 | MX2 | | 0 | 1 | 1 | 0.00601 | Not Detected but | Confirmed |
| | | | | | | | Confirmed | |
| | | | 22 | 65 | 33 | | | |

| | | |
|---|---|---|
| Detected by Filter and p < 0.05 by | 13 | 26 |
| RT-PCR | | |
| Detected by Filter and p > 0.05 by | 2 | 7 |
| RT-PCR | | |
| Detected by Filter but Not Tested by | 7 | 32 |
| RT-PCR | | |
| Not Detected by Filter but | 13 | 0 |
| Confirmed by RT-PCR | | |
| Not Detected by Filter and Not | 30 | 0 |
| Confirmed by RT-PCR | | |

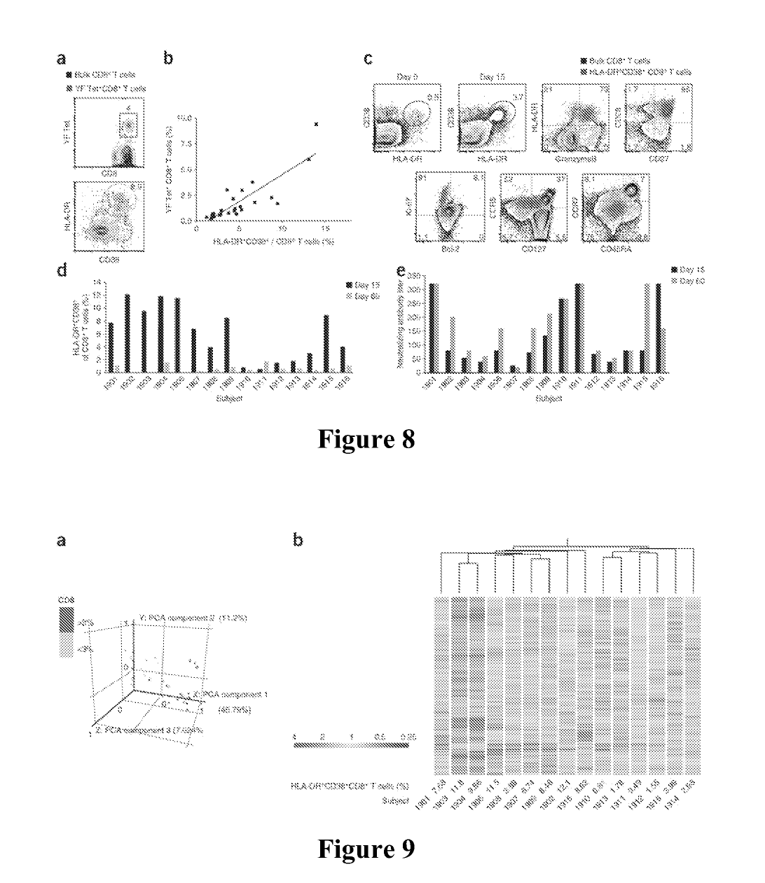

This table compares the genes identified using 2 independent strategies: a ‘pre-filtering strategy,’ and a strategy of testing the entire database with ANOVA without pre-filtering. In the ‘pre-filtering’ strategy, genes with normalized Log2 transformed fold change gene expression values >0.5 or <0.5 in >60% of the subjects at days 3 or 7 were identified, and then tested for statistical significance by ANOVA adjusted with the Benjamini and Hochberg False Discovery Rate method with a cutoff of 0.05 in Genespring. This analysis revealed a set of 65 genes that were commonly induced in both Trials 1 and 2 (indicated by a ‘1’ in the column entitled ‘Pre-Filtered’). Of these 65 genes, the ones that were chosen for validation by RT-PCR are indicated by a ‘1’ in the column entitled ‘RT-PCR’ The RT-PCR P-values, and the results of this validation process, are indicated in the columns ‘RT-PCR’, and ‘Pre-Filtered versus RT-PCR’, respectively. In the second strategy not involving pre-filtering, the entire dataset was tested using ANOVA (column entitled ‘ANOVA only’), and this yielded 22 genes which were a subset of the 65 genes identified via the pre-filtering method. The majority of these genes were confirmed by RT-PCR (column entitled ‘ANOVA vs RT-PCR’). Importantly, many genes that were not included in this subset of 22 genes were also confirmed by RT-PCR. Furthermore, the genes encoding CD38 and IP-10, which were demonstrated to be expressed at the protein level (FIG. 8 and FIG. 1), were not present amongst the 22 genes. Thus, while this second method of analysis omitting the pre-filtering step can result in a more rigorous statistical analysis, it may be too stringent and exclude potentially biologically relevant genes.

Microarray analysis of total PBMCs revealed a molecular signature comprised of genes involved in innate sensing of viruses and antiviral immunity in most of the vaccinees. Of note, both studies demonstrated that there was robust induction of a network of genes encoding innate sensing receptors such as TLR7, RIG-I, and melanoma differentiation-associated gene 5 (MDA5), and the cytoplasmic receptors for members of the 2′,5′-oligoadenylate synthetase family, as well as transcription factors that regulate the expression of type I IFNs, IFN regulator factor 7 (IRF7) and signal transducer and activator of transcription 1 (STAT1)^{30,31}. Consistent with this, YF-17D was also shown to signal via RIG-I and MDA5³⁰. Furthermore, there was induction of the gene encoding the RIG-I like RNA helicase, LGP2^{30,31}, which is a negative regulator of RIG-1- and MDA5-mediated responses³⁴. Furthermore, genes encoding proteins in the complement pathway (e.g. C1qB) and the inflammasome were induced. By visualizing these gene networks, a group of transcription factors, including IRF7, STAT1 and ETS2, were identified as key regulators of the early innate immune response to the YF-17D vaccine^{30,31}. Importantly, there was a persistent upregulation of this anti-viral gene signature for more than 2 weeks post vaccination³⁰, presumably reflecting the ongoing stimulation of innate immune cells in response to viral replication, which peaks at 7 days^{2,3}. This signature reflects that fact that vaccination with YF-17D results in a live viral infection, and it is likely that other viruses that stimulate potent immune responses induce a similar signature. However the question of whether the pathogenic Asibi strain also induces a similar signature remains to be determined. Thus, to what extent this signature simply mimics a viral infection versus whether it has any relevance for the adaptive immune response is unclear. Indeed, there was no correlation between the induction of these genes and the magnitude of the CD8 T⁺ cell or neutralizing antibody response.

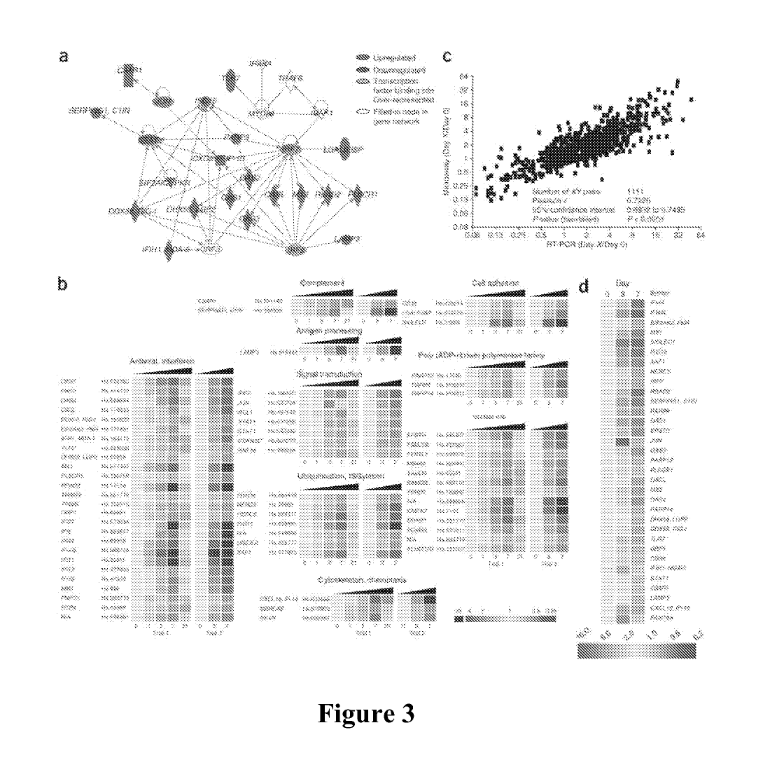

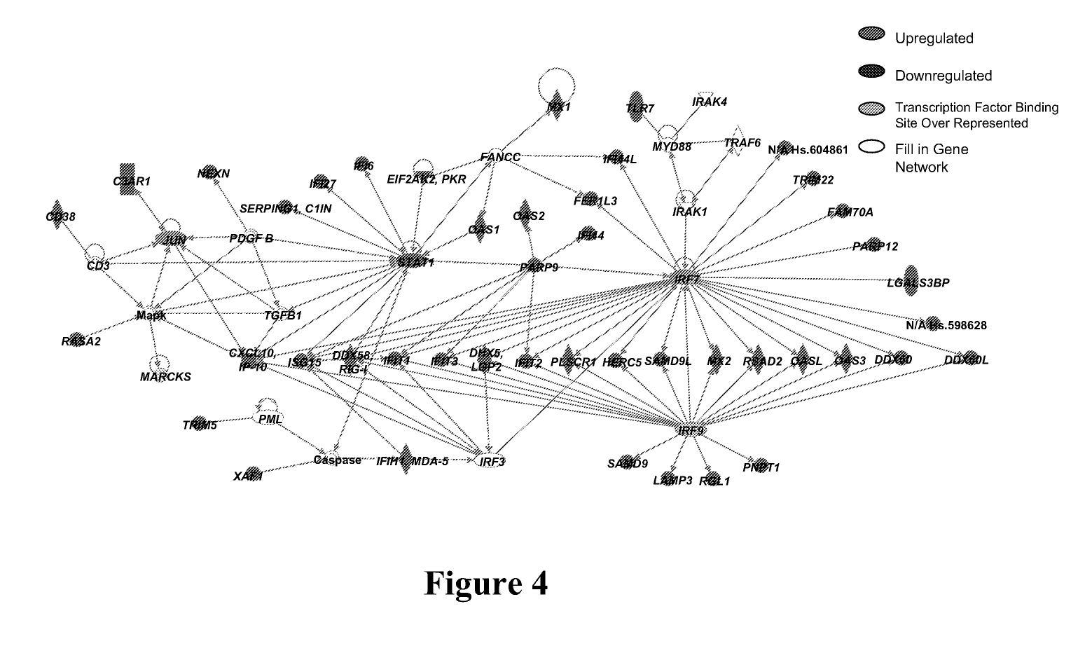

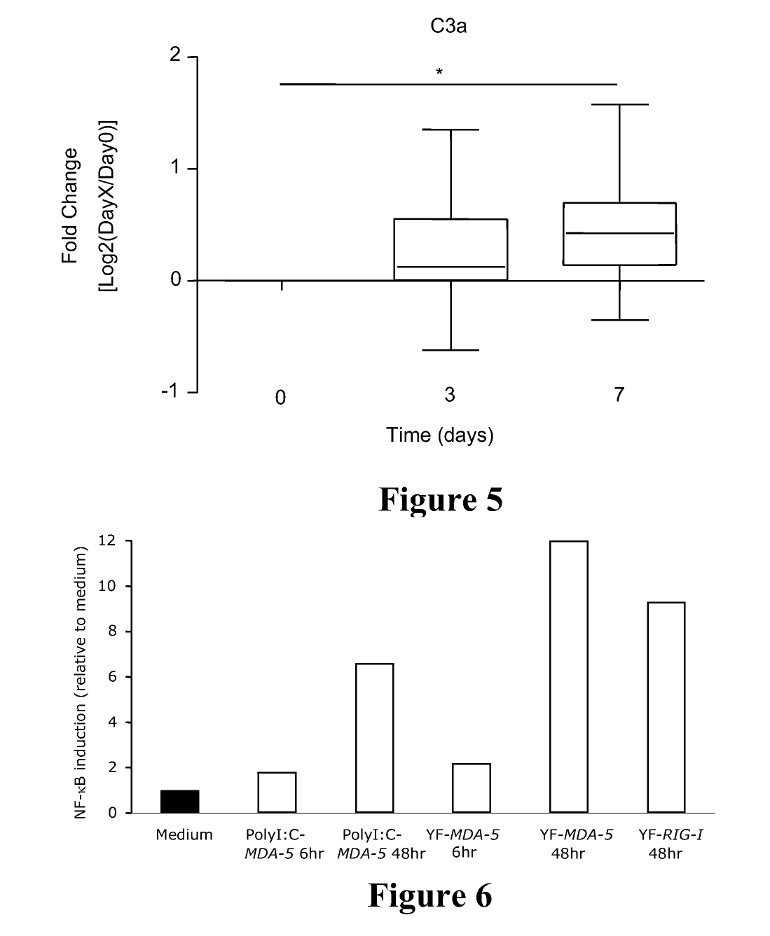

Using the DAVID Bioinformatics Database, the Gene Ontology terms associated with the doubly confirmed set of 65 genes were analyzed, which revealed an enrichment of genes related to various immunological responses, cell motility and biopolymer metabolism (FIG. 2b). Those genes were then imported into TOUCAN for transcription factor binding site (TFBS) analysis, and 44 out of the 65 genes were recognized. The TFBSs found to have statistically over-represented frequencies included the interferon-stimulated response element (ISRE), interferon regulatory factor 7 (IRF7) binding site and sterol regulatory element-binding protein 1 (SREBF1) binding site (Table 2). Visualization of gene networks with Ingenuity Pathways Analysis supplemented with the TOUCAN transcription factor motif information revealed a closely interacting network of 50 interferon and antiviral genes, including IRF7, OAS1, OAS2, OAS3 and OASL; genes involved in viral recognition, including TLR7, DDX58 (RIG-I), IFIH1 (MDA-5), DHX58 (LGP2) and EIF2AK2 (PKR); and genes mediating antiviral immunity, such as CXCL10 (IP-10), MX1, and the complement genes SERPING1 (C1IN) and C3AR1 (FIGS. 3a and 4). Consistent with this, C3a, a product of the classical, alternative, and mannan-binding lectin complement enzymatic pathways and an anaphylatoxin with chemotactic properties, was increased at day 7 (FIG. 5). Furthermore, YF-17D was observed to signal through RIG-I and MDA-5 to induce NF-κB activation (FIG. 6).

TABLE 2
(Supplemental Table 2) Two transcription factors
induced by YF-17D in two independent trials.

| Description | Feature Name | Factor Name | N | P-value |
|---|---|---|---|---|
| Interferon- | M00258- | IRF9 | 27 | 2.49E−06 |
| Stimulated | V$ISRE_01 | | | |
| Response Element | | | | |
| Interferon | M00453- | IRF7 | 30 | 7.64E−04 |
| Regulatory | V$IRF7_01 | | | |
| Factor 7 | | | | |
| Sterol Regulatory | M00220- | SREBF1 | 15 | 0.005390915 |
| Element-Binding | V$SREBP1_01 | | | |
| Protein 1 | | | | |

The 65 genes which were found to be induced by YF-17D in FIG. 3b were imported into TOUCAN for transcription factor binding site (TFBS) analysis, and 44 out of the 65 genes were recognized by TOUCAN. The TRANSFAC v7.0 database of eukaryotic transcription factors was used as the reference for transcription factor binding site motifs. Binding site motifs were scanned in the DNA sequence 2000 bases upstream through 200 bases downstream flanking the first exon of each gene with a double prior of 0.1 and the genomic background noise model based on the third order Markov Model for the Human Eukaryotic Promoter Databse. The TFBSs found to have a statistically overrepresented frequencies including: interferon-stimulated response element (ISRE), interferon regulatory factor 7 (IRF7), and sterol regulatory element-binding protein 1 (SREBF1).

Using additional bioinformatics approaches, we identified gene signatures that did correlate with the magnitude of antigen-specific CD8⁺ T cell responses and antibody titres. To evaluate the actual predictive ability of this signature, we determined whether the gene signatures could predict the magnitude of the CD8⁺ T cell or B cell response in individuals from a second YF-17D vaccine trial. We observed that several signatures for CD8⁺ T cell responses from the first trial were predictive with up to 90% accuracy in the second trial and vice versa. Interestingly, two genes, solute carrier family 2, member 6 (SLC2A6) and eukaryotic translation initiation factor 2α kinase 4 (EIF2AK4) were present in the predictive signatures identified using two independent bioinformatics prediction models. SLC2A6 belongs to a family of membrane proteins that regulate glucose transport and glycolysis in mammalian cells. EIF2AK4 has an important role in the integrated stress response, and regulates protein synthesis in response to environmental stresses by phosphorylating elongation initiation factor 2α (eIF2α). The translation of constitutively expressed proteins is terminated by redirection of their mRNAs from. Consistent with this, YF-17D induced the phosphorylation of eIF2α and the formation of stress granules. Moreover, several other genes involved in the stress response pathway, including calreticulin, protein disulfide isomerase, glucocorticoid receptor and JUN, correlated with the CD8⁺ T cell response. Recent work has shown an antiviral effect of EIF2AK4 against RNA viruses, but the affect of this on adaptive immune responses is not known. It is thus tempting to speculate that the induction of the integrated stress response in innate immune cells might regulate the adaptive immune response to YF-17D, and perhaps other vaccines or microbial stimuli. With respect to antibody responses, TNF receptor superfamily, receptor 17 (TNFRSF17), which is a receptor for B cell-activating factor (BAFF), was shown to be a key gene in the predictive signatures. BAFF is thought to optimize B cell responses to B cell receptor- and TLR-dependent signaling. Thus, taken together these studies provide a global description of the innate and adaptive immune responses that are induced by YF-17D vaccination and highlight the complexity of the innate immune response that is required for the induction of long-lasting immune protection.

To depict gene expression in an organized fashion, those 65 genes were first categorized into sub-lists based on gene comment and summary information available through DAVID. The kinetics of expression of these gene sub-lists are presented as heat maps of baseline normalized expression (FIG. 3b). There was good agreement between trial 1 and trial 2 on the relative change of expression of each gene. Some genes changed as early as days 1 and 3, but the peak change for most genes was reached on day 7. The largest category contained genes with a clear role in interferon and innate antiviral responses, such as IRF7 and STAT1. Other notable categories included genes in the complement pathway and ubiquitination and/or ISGylation (modification of proteins by addition of interferon stimulatory gene (ISG) products). For an independent verification of these genes, 10 day 3/day 0 and 15 day 7/day 0 changes in trial 1 were assayed by RT-PCR. A significant correlation (P<0.0001) existed between the microarray data and RT-PCR results (FIG. 3c and Table 3). To test whether the RT-PCR data would independently measure significant changes in gene expression after YF-17D vaccination, a subset of 33 genes of greatest interest from the original microarray data were tested for relative RT-PCR expression by one-way ANOVA over time. Of the 33 genes, 26 had a P-value less than 0.05, confirming the microarray data (FIG. 3d).

TABLE 3
RT-PCR confirmation of the genes induced by YF-17D.

| Symbol | Gene ID | TaqMan Assay | P-value |
|---|---|---|---|
| IFI44 | Hs.8231 6 | Hs001 97427_m1 | 0.0000 |
| IFI44L | Hs.389724 | HS001991 15_m1 | 0.0000 |
| EIF2AK2, PKR | Hs.131431 | Hs00169345_m1 | 0.0038 |
| MX1 | Hs.51 7307 | Hs001 82073_m1 | 0.0001 |
| SIGLEC1 | Hs.31 869 | Hs00224991_m1 | 0.0004 |
| ISG15 | Hs.458485 | Hs001 92713_m1 | 0.0001 |
| XAF1 | Hs.441975 | Hs0021 3882_m1 | 0.0001 |
| HERC5 | Hs.26663 | Hs001 80943_m1 | 0.0002 |
| IRF7 | Hs.166120 | Hs00242190_g1 | 0.0001 |
| RSAD2 | Hs.1 751 8 | Hs0036981 3_m1 | 0.0006 |
| SERPING1, C1IN | Hs.384598 | Hs001 63781_m1 | 0.0084 |
| PARP9 | Hs.51 8200 | Hs00230231_m1 | 0.0012 |
| OAS1 | Hs.524760 | Hs00242943_m1 | 0.0012 |
| EPSTI1 | Hs.546467 | Hs00264424_m1 | 0.0007 |
| JUN | Hs.525704 | Hs99999141_s1 | 0.0001 |
| OAS3 | Hs.528634 | Hs001 96324_m1 | 0.0019 |
| PARP12 | Hs.1 2646 | Hs00224241_m1 | 0.0066 |
| PLSCR1 | Hs.1 30759 | Hs00275514_m1 | 0.0006 |
| OASL | Hs.1 18633 | Hs00388714_m1 | 0.0005 |
| MX2 | Hs.926 | Hs001 5941 8_m1 | 0.0060 |
| OAS2 | Hs.414332 | Hs00159719_m1 | 0.0057 |
| PARP14 | Hs.51 8203 | Hs00393814_m1 | 0.984 |
| DHX58, LGP2 | Hs.5591 8 | Hs00225561_m1 | 0.0402 |
| DDX58, RIG-I | Hs.1 90622 | Hs00204833_m1 | 0.0160 |
| TLR7 | Hs.443036 | Hs001 52971_m1 | 0.0409 |
| GBP1 | Hs.62661 | Hs0026671 7_m1 | 0.0707 |
| CD38 | Hs.479214 | Hs00277045_m1 | 0.0311 |
| IFIH1, MDA-5 | Hs.1 631 73 | Hs00223420_m1 | 0.0022 |
| STAT1 | Hs.651258 | Hs00234829_m1 | 0.0528 |
| C3AR1 | Hs.591 148 | Hs00269693_s1 | 0.0732 |
| LAMP3 | Hs.51 8448 | Hs001 80880_m1 | 0.3765 |
| CXCL10, IP-10 | Hs.632586 | Hs001 71 042_m1 | 0.2288 |
| FAM70A | Hs.437563 | Hs00215705_m1 | 0.4147 |

Of the 65 genes induced in most vaccinees (FIG. 3), 33 genes were selected for RT-PCR analysis. Ten day 3 versus day 0 and 15 day 7 versus day 0 time points were assayed from the 15 subjects in Trial 1. This revealed that 26 genes also have significant modulation as measured by RT-PCR. The P-values are testing the result of ANOVA on the RT-PCR data for the fold changes on days 0, 3, and 7.

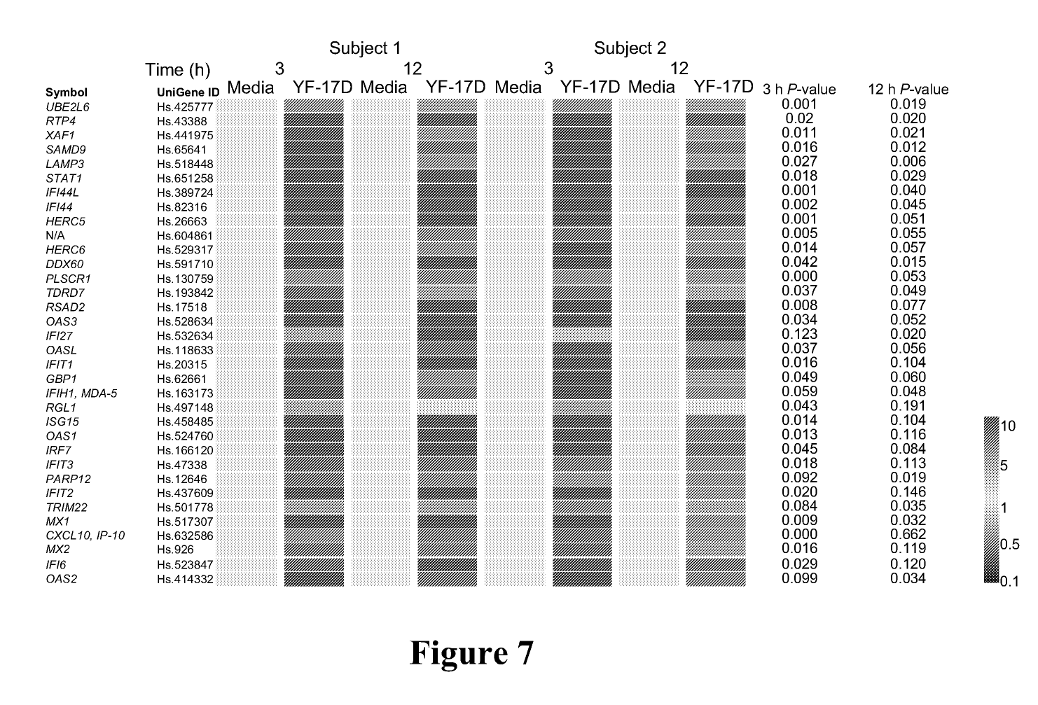

Induction of this gene signature in response to YF-17D could have resulted from recruitment of specific cell types containing abundant transcripts for these genes, rather than de novo induction of gene expression. To determine whether YF-17D induced de novo expression of genes in PBMCs, PBMCs were stimulated in vitro with YF-17D for 3 or 12 h and then evaluated gene expression. Of the 65 genes induced in vivo, 34 were reproducibly and significantly induced (P<0.05; FIG. 7). This result demonstrated that YF-17D was able to modulate the expression of these genes in a fixed population of cells. Taken together, this analysis revealed that the innate immune response to YF-17D vaccine was characterized by induction of IP-10 and IL1A (IL-1α) (FIGS. 1a and b), upregulation of CD86 on DCs and monocytes (FIG. 1c), induction of a ‘network’ of genes mediating interferon-related antiviral responses (FIG. 3 and FIG. 4), and complement activation (FIG. 5)

(2) Variable CD8+ T Cell and Antibody Responses

Next the antigen-specific CD8+ T cell response and neutralizing antibody titers induced by vaccination were evaluated. During the response to vaccination with YF-17D, activated CD8+ T cells transiently upregulate HLA-DR, CD38 and Ki-67 (a protein expressed during the cell cycle) and downregulate the antiapoptotic protein Bcl-2, and that the peak of expansion occurs at 2 weeks. During this study, a newly identified HLA-A0201-specific epitope in YF-17D was mapped; tracking CD8+ T cells by in vitro flow cytometry using tetramers made with this epitope revealed that antigen-specific CD8+ T cells appeared at the same time as the HLA-DR+CD38+ population, and they constituted a subset of HLA-DR+CD38+ cells at 2 weeks after vaccination (FIG. 8a). Also, the magnitude of the epitope-specific CD8+ T cell responses in HLA-A2+ vaccinees was directly proportional (r2=0.724, P<0.0001) to the size of their HLA-DR+CD38+ population (FIG. 8b). Together these data support the use of HLA-DR and CD38 to measure the magnitude of the YF-17D-specific CD8+ T cell response.

In addition, these CD8+ T cells expressed markers of T cell activation and function typical of effector T cells, including granzyme B, CD27, CD28 and CCR5 (FIG. 8c) and low abundances of CD45RA, CCR7 and CD127, when compared to naive CD8 T cells (FIG. 8c). Analysis of CD8+ T cell activation by percentage of CD38+ HLA-DR+ cells at day 15 after vaccination showed, unexpectedly, that even with this highly effective vaccine, immune responses varied among individuals by more than tenfold (FIG. 8d). Notably, the magnitude of the CD8+ T cell response at day 15 had a strong correlation with the magnitude of the response at later time points, such as day 30 (Pearson r=0.9135; P=0.0001 (two-tailed)). Similarly, the neutralizing antibody titers also varied considerably among the 15 individuals (FIG. 8e).

(3) Signatures that Predict Antigen-Specific CD8+ T Cell Responses

Notably, neither the induction of IP-10 or IL1A (IL-1α) nor the upregulation of CD86 on antigen-presenting cells (FIG. 1) correlated with the magnitude of the CD8+ T cell response. Furthermore, there was no correlation between the expression of the genes identified in the gene expression analysis described above (FIG. 3a) and the magnitude of the CD8+ T cell response. Therefore, an early gene signature that correlated with the magnitude of the CD8+ T cell response in the 15 individuals in the first trial was sought. 839 genes were identified that correlated with the magnitude of the CD8+ T cell response (Methods and FIG. 9). As indicated by analysis in DAVID, these genes were largely associated with metabolism and immunological responses (Table 4). To visualize how well the genes identified by the relative expression and P-value cutoffs sorted the subjects in terms of CD8+ T cell responses, an unsupervised principal component analysis was performed. The genes segregated the subjects into two subgroups, with an activated CD8+ T cell cutoff of 3% CD38+HLA-DR+ (FIG. 9a). GeneSpring's standard correlation with average linkage hierarchical clustering analysis confirmed that the subjects segregated into two groups on the basis of gene expression and the cutoff point was approximately 3% CD8+ T cell activation (FIG. 9b).

TABLE 4
Genomic signatures that correlate with the
magnitude of the CD8⁺ T cell response

| Gene ontology term | Count | Percentage | P-value |
|---|---|---|---|
| Cellular metabolism | 292 | 42.9 | 1.40 × 10⁻⁴ |
| Primary metabolism | 281 | 41.3 | 3.60 × 10⁻⁴ |
| Macromolecule metabolism | 183 | 26.9 | 1.10 × 10⁻³ |
| Protein localization | 34 | 5 | 1.50 × 10⁻³ |
| Response to pest, | 30 | 4.4 | 8.10 × 10⁻³ |
| pathogen or parasite | | | |
| Response to other organism | 31 | 4.6 | 1.00 × 10⁻² |
| Establishment of | 118 | 17.4 | 1.30 × 10⁻² |
| localization | | | |
| Viral genome replication | 4 | 0.6 | 2.70 × 10⁻² |
| Regulation of cellular | 125 | 18.4 | 3.50 × 10⁻² |
| physiological process | | | |
| Cell organization | 62 | 9.1 | 3.60 × 10⁻² |
| and biogenesis | | | |
| Transport | 106 | 15.6 | 3.80 × 10⁻² |
| Regulation of metabolism | 95 | 14 | 6.80 × 10⁻² |
| Nitrogen compound metabolism | 19 | 2.8 | 8.20 × 10⁻² |
| Negative regulation | 29 | 4.3 | 8.40 × 10⁻² |
| of physiological process | | | |
| Response to wounding | 19 | 2.8 | 8.50 × 10⁻² |

Genes identified in FIG. 9 were analyzed by DAVID for associations with particular Gene Ontology terms. The P-values refer to how significant an association a particular gene ontology term has with the gene list.

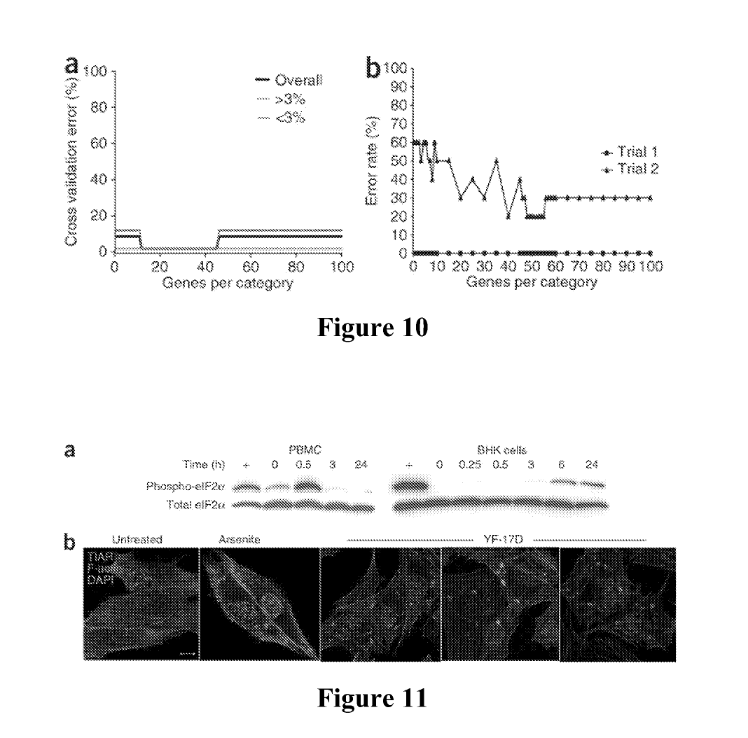

However, the real test of such a signature is the extent to which it can truly predict the immune response in an independent trial. To determine whether the gene signature identified in trial 1 could predict the magnitude of the CD8+ T cell response in trial 2 (and vice versa, two independent classification methods, called classification to nearest centroid (ClaNC) and discriminant analysis via mixed integer programming (DAMIP) were used. ClaNC has been previously shown to successfully develop predictive transcriptional cancer models. Using the ClaNC model, the minimum number of genes in the signature of 839 genes (FIG. 10) required to correctly classify vaccinees was determined in trial 1 into the high (>3%) and low (<3%) CD8+ T cell responders (FIGS. 10a and b). This unsupervised model was first developed by plotting the error rates in this classification versus the number of genes (FIG. 10a). Zero errors in cross-validations were obtained with 10 to 45 genes per CD8+ T cell category (FIG. 10a). Next, the signature identified in trial 1 was used to classify the vaccinees in trial 2 into high (>3%) versus low (<3%), CD8+ T cell responders (FIG. 10b). Using less than 20 genes yielded error rates oscillating around 50%, which is no better than would be produced by chance; increasing the number of genes in the models stabilized the overall error rates at 20% (FIG. 10b). A minimum subset of 48 genes was needed to reach the minimum error rate (FIG. 10b and Table 5); the requirement for as many as 48 genes to accurately classify 15 subjects indicated overtraining, however.

TABLE 5
The genes validated by ClaNC as being predictive
of CD8+ T cell responses from FIG. 10

| Symbol | Gene Name | UniGene ID | GeneBank | Day |
|---|---|---|---|---|
| C1QB | Complement component 1, q | Hs.8986 | CA307782 | 3 |
| | subcomponent, B chain | | | |
| E!F2AK4 | Eukaryotic translation initiation factor | Hs.412102 | BM978043 | 7 |
| | 2 alpha kinase 4 | | | |
| MEF2A | MADS box transcription enhancer | Hs.268675 | Y1 6312 | 7 |
| | factor 2, polypeptide A | | | |
| SLC2A6 | Solute carrier family 2, member 6 | Hs.244378 | AJ01 1372 | 7 |
| ALDH16A1 | Aldehyde dehydrogenase 16 family, | Hs.355398 | BU741307 | 7 |
| | member A1 | | | |
| ALDH3B1 | Aldehyde dehydrogenase 3 family, | Hs.523841 | BC014168 | 3 |
| | member B1 | | | |
| ASGR2 | Asialoglycoprotein receptor 2 | Hs.16247 | CR594935 | 3 |
| ASGR2 | Asialoglycoprotein receptor 2 | Hs.16247 | CR594935 | 7 |
| ATP6V1E1 | ATPase, H+ transporting, lysosomal | Hs.51 7338 | AW804839 | 3 |
| | 31 kDa,V1 subunit E1 | | | |
| B!RC3 | Baculoviral IAP repeat-containing 3 | Hs.127799 | BQ004306 | 7 |
| BN!P3L | BCL2/adenovirus E1 B 19 kDa | Hs.131226 | NM_004331 | 7 |
| | interacting protein 3-like | | | |
| BCKDK | Branched chain ketoacid | Hs.513520 | AF026548 | 3 |
| | dehydrogenase kinase | | | |
| CAMKK2 | Calcium/calmodulin-dependent | Hs.297343 | NM_1 72226 | 3 |
| | protein kinase kinase 2, beta | | | |
| CALR | Calreticulin | Hs.515162 | BM806569 | 3 |
| CRAT | Carnitine acetyltransferase | Hs.12068 | AI809851 | 3 |
| CTSB | Cathepsin B | Hs.520898 | NM_001908 | 3 |
| CD69 | CD69 molecule | Hs.208854 | AU309880 | 3 |
| N/A | CDNA clone IMAGE: 5271 145 | Hs.385760 | BC038776 | 7 |
| N/A | CDNA FLJ20387 fis, clone | Hs.636439 | AK000394 | 7 |
| | KAIA4452 | | | |
| N/A | CDNA: FLJ20905 fis, clone | Hs.61 2877 | AK024558 | 3 |
| | ADSE00244 | | | |
| CENPB | Centromere protein B, 80 kDa | Hs.516855 | BM703471 | 3 |
| CXCR6 | Chemokine (C-X-C motif) receptor 6 | Hs.34526 | CR624554 | 3 |
| CXCR7 | Chemokine (C-X-C motif) receptor 7 | Hs.471 751 | BX1 11686 | 3 |
| CXCR7 | Chemokine (C-X-C motif) receptor 7 | Hs.471 751 | BX1 11686 | 7 |
| DEFA4 | Defensin, alpha 4, corticostatin | Hs.591391 | NM_001925 | 7 |
| EM!L!N2 | Elastin microfibril interfacer2 | Hs.532815 | AF270513 | 7 |
| ETV3 | Ets variant gene 3 | Hs.352672 | AF218540 | 3 |
| E!F4G3 | Eukaryotic translation initiation | Hs.467084 | AF012072 | 7 |
| | factor 4 gamma, 3 | | | |
| FBXO15 | F-box protein 15 | Hs.465411 | DB522515 | 7 |
| GPR18 | G protein-coupled receptor 18 | Hs.631 765 | AW57481 1 | 7 |
| GBGT1 | Globoside alpha-1,3-N- | Hs.495419 | CR622726 | 3 |
| | acetylgalactosaminyltransferase 1 | | | |
| GAA | Glucosidase, alpha; acid | Hs.1437 | AL043560 | 3 |
| GAS2L1 | Growth arrest-specific 2 like 1 | Hs.322852 | BC001 782 | 3 |
| HEATR3 | HEAT repeat containing 3 | Hs.647381 | AW802598 | 7 |
| HBA 1 | Hemoglobin, alpha 1 | Hs.449630 | AA331275 | 7 |
| HBB | Hemoglobin, beta | Hs.523443 | BP424559 | 3 |
| HBB | Hemoglobin, beta | Hs.523443 | BP424559 | 7 |
| HBZ | Hemoglobin, mu | Hs.647389 | CR597411 | 7 |
| HTRA4 | HtrA serine peptidase 4 | Hs.322452 | AL574735 | 3 |
| FLJ10847 | Hypothetical protein FLJ10847 | Hs.232054 | AI014423 | 7 |
| SLC47A 1 | Hypothetical protein LOC731 157 | Hs.551062 | AF150372 | 7 |
| !MPDH1 | IMP (inosine monophosphate) | Hs.534808 | BU687473 | 3 |
| | dehydrogenase 1 | | | |
| JUN | Jun oncogene | Hs.525704 | NM _002228 | 3 |
| C8orf82 | Chromosome 8 open reading frame 82 | Hs.105685 | AA532638 | 3 |
| C8orf82 | Chromosome 8 open reading frame 82 | Hs.105685 | AA532638 | 7 |
| MYL4 | Myosin, light chain 4, alkali; atrial, | Hs.463300 | AJ706934 | 7 |
| | embryonic | | | |
| NANS | N-acetylneuraminic acid synthase | Hs.522310 | AA639295 | 7 |
| | (sialic acid synthase) | | | |
| NRGN | Neurogranin (protein kinase C | Hs.524116 | NM_006176 | 3 |
| | substrate, RC3) | | | |
| NAPRT1 | Nicotinate | Hs.493164 | BM674162 | 3 |
| | phosphoribosyltransferase domain | | | |
| | containing 1 | | | |
| NAPRT1 | Nicotinate | Hs.493164 | BM674162 | 7 |
| | phosphoribosyltransferase domain | | | |
| | containing 1 | | | |
| NP | Nucleoside phosphorylase | Hs.75514 | AW519082 | 3 |
| NUDT14 | Nudix (nucleoside diphosphate | Hs.526432 | CA775837 | 3 |
| | linked moiety X)-type motif 14 | | | |
| PNPLA6 | Patatin-like phospholipase domain | Hs.631863 | DN993154 | 3 |
| | containing 6 | | | |
| PRAM1 | PML-RARA regulated adaptor | Hs.46581 2 | AW1 35236 | 3 |
| | molecule 1 | | | |
| PRAM1 | PML-RARA regulated adaptor | Hs.46581 2 | AW1 35236 | 7 |
| | molecule 1 | | | |
| RAB8B | RAB8B, member RAS oncogene | Hs.389733 | NM_016530 | 3 |
| | family | | | |
| RGS1 | Regulator of G-protein signalling 1 | Hs.75256 | BU783195 | 3 |
| NDRG2 | Selenium binding protein 1 | Hs.632460 | CN2621 11 | 7 |
| STK17A | Serine/threonine kinase 17a | Hs.268887 | NM_004760 | 3 |
| | (apoptosis-inducing) | | | |
| SMARCD3 | SMARC, subfamily d, member 3 | Hs.647067 | CA449683 | 3 |
| SLC16A5 | Solute carrier family 16, member 5 | Hs.592095 | AI953766 | 3 |
| SLC2A6 | Solute carrier family 2, member 6 | Hs.244378 | AJ01 1372 | 3 |
| SLC25A13 | Solute carrier family 25, member 13 | Hs.489190 | AJ496569 | 7 |
| | (citrin) | | | |
| SLC39A11 | Solute carrier family 39 (metal ion | Hs.221 127 | BQ01 7291 | 7 |
| | transporter), member 11 | | | |
| SAT2 | Spermidine/spermine N1- | Hs.10846 | CK821652 | 3 |
| | acetyltransferase 2 | | | |
| SAT2 | Spermidine/spermine N1- | Hs.10846 | CK821652 | 7 |
| | acetyltransferase 2 | | | |
| SPON2 | Spondin 2, extracellular matrix | Hs.302963 | DB319294 | 7 |
| | protein | | | |
| N/A | Transcribed locus | Hs.642649 | BE464165 | 3 |
| TBC1D7 | TBC1 domain family, member 7 | Hs.484678 | BF111612 | 3 |
| TEP1 | Telomerase-associated protein 1 | Hs.508835 | CD623678 | 3 |
| THAP1 1 | THAP domain containing 11 | Hs.632200 | BP395356 | 3 |
| ADSSL1 | Transcribed locus | Hs.375179 | AA927922 | 7 |
| ZEB1 | Transcribed locus | Hs.593418 | AI806174 | 7 |
| ASGR2 | Transcribed locus | Hs.595979 | H47090 | 7 |
| ASGR2 | Transcribed locus | Hs.595979 | H47090 | 3 |
| CPEB3 | Transcribed locus | Hs.60321 8 | AI 123721 | 7 |
| N/A | Transcribed locus | Hs.604822 | AI370631 | 3 |
| N/A | Transcribed locus | Hs.607204 | AI862844 | 3 |
| N/A | Transcribed locus | Hs.649837 | AA528126 | 3 |
| N/A | Transcribed locus | Hs.651406 | AA600976 | 3 |
| N/A | Transcribed locus | Hs.652017 | CA844149 | 3 |
| N/A | Transcribed locus | Hs.652017 | CA844149 | 7 |
| N/A | Transcribed locus | Hs.652922 | CA313785 | 3 |
| N/A | Transcribed locus | Hs.604290 | AI281031 | 7 |
| TCEAL4 | Transcription elongation factor A | Hs.194329 | BF718552 | 3 |
| | (SII)-like 4 | | | |
| TMEM176A | Transmembrane protein 176A | Hs.6471 16 | BM663079 | 3 |
| TMOD1 | Tropomodulin 1 | Hs.494595 | AK095748 | 7 |
| TUFM | Tu translation elongation factor, | Hs.12084 | AA983218 | 3 |
| | mitochondrial | | | |
| TNFSF14 | Tumor necrosis factor (ligand) | Hs.129708 | AY028261 | 3 |
| | superfamily, member 14 | | | |
| FERMT3 | UNC-1 12 related protein 2 | Hs.180535 | BF975449 | 3 |
| ULK2 | Unc-51-like kinase 2 (C. elegans) | Hs.168762 | NM_014683 | 7 |
| WDR40A | WD repeat domain 40A | Hs.651 274 | AA4461 17 | 7 |
| ZFP82 | Zinc finger protein 545 | Hs.558734 | BU618382 | 3 |
| ZNF606 | Zinc finger protein 606 | Hs.6521 13 | BM713422 | 3 |
| ZSWIM5 | Zinc finger, SWIM-type containing 5 | Hs.135673 | BQ448086 | 7 |
| ZYX | Zyxin | Hs.490415 | CB160586 | 3 |

Therefore, a second approach the DAMIP classification model, a general-purpose optimization-based predictive modeling framework and computational engine, was used which is a very powerful supervised-learning classification approach in predicting various biomedical and biobehavioral phenomena, owing to the universal consistency of the resulting classification rules and their ability to classify with high prediction accuracy even among small training sets. Furthermore, DAMIP is a discrete support vector machine coupled with a powerful feature selection module, and it has been proven in earlier studies to produce superior classification accuracy when compared to traditional quadratic or linear discriminant analysis. The DAMIP model was trained using trial 1 to obtain an unbiased estimate of correct classification. This was then followed by a blind test to predict the response of the subjects in trial 2. Specifically, trial 1 consisted of ten subjects in the high group and five in the low group, and trial 2 consisted of five subjects in the high group and five in the low group (FIGS. 9a and b).

DAMIP allows the user to input the desired misclassification rate, and the classification system then returns predictive rules (each with the associated set of discriminatory patterns) that satisfy the input misclassification rate. In the analysis, setting the training error rate to be 20%, eight independent signature (discriminatory) sets, each associated with a predictive rule, were generated (Table 6). Each predictive rule was generated by a signature set with only two or three discriminatory genes, and each produced an unbiased estimate of 93% correct classification in tenfold cross-validation (Table 6). Using these predictive rules generated from trial 1, blind tests on trial 2 were performed. To evaluate the consistency of the classification rules, in addition to single-fold blind test tenfold blind tests were conducted. In the singlefold prediction, the prediction accuracy of trial 2 status was at least 80% among all rules produced by these eight independent signature sets, with some signatures reaching blind prediction rates of 90% (Table 6). The tenfold blind prediction showed a similar trend, with prediction accuracies ranged from 80-88%. Examination of each single-fold and tenfold pair revealed that the prediction rates between them were within 5%, thus validating that each classification rule obtained from trial 1 was highly consistent and stable in the trial 2 blind-prediction process. Several genes, including EIF2AK4 (A000827) and SLC2A6, were present in several signature sets of the DAMIP model and were also present in the ClaNC model (Table 5). Notably, training on trial 2 and testing on trial 1 yielded several predictive signatures, which also contained EIF2AK4 and SLC2A6 (Table 6).

TABLE 6
Genomic signatures that predict the magnitude of the CD8⁺ T cell responses using the DAMIP model

| | | | DAMIP model predictive signatures | | | | | | | | | | | |
|---|---|---|---|---|---|---|---|---|---|---|---|---|---|---|
| | Gene | | Train on trial 1, test on trial 2 | | | | | | | | Train on trial 2, test on trial 1 | | | |
| Gene name | symbol | Gene ID | 1 | 2 | 3 | 4 | 5 | 6 | 7 | 8 | 1 | 2 | 3 | 4 |
| Solute carrier | SLC2A6 | Hs.244378 | X | | X | X | X | X | X | X | X | X | X | |
| family 2 | | Day 7 | | | | | | | | | | | | |
| (facilitated glucose | | | | | | | | | | | | | | |
| transporter), member 6 | | | | | | | | | | | | | | |
| Eukaryotic translation | EIF2AK4 | Hs.412102 | X | X | X | | X | | X | X | | | | |
| initiation factor 2 alpha | | Day 7 | | | | | | | | | | | | |
| kinase 4 | | | | | | | | | | | | | | |
| Integrin, alpha L (antigen | ITGAL/LFA-1 | Hs.174103 | | | X | | | | | | | | | X |
| CD11A) | | Day 7 | | | | | | | | | | | | |
| C-terminal binding | CTBP1 | Hs.208597 | | | | X | | | | | | | | |
| protein 1 | | Day 7 | | | | | | | | | | | | |
| Tyrosine 3- | YWHAE | Hs.513851 | | | | X | X | | | | | | | |
| monooxygenase/tryptophan | | Day 3 | | | | | | | | | | | | |
| 5-monooxygenase | | | | | | | | | | | | | | |
| activation protein | | | | | | | | | | | | | | |
| Transcribed locus | | Hs.619443 | | | | | | X | | | X | X | X | X |
| | | Day 7 | | | | | | | | | | | | |
| Protein phosphatase 1, | PPP1R14A | Hs.631569 | | | | | | | X | | | | | |
| regulatory (inhibitor) | | Day 3 | | | | | | | | | | | | |
| subunit 14A | | | | | | | | | | | | | | |
| Family with sequence | FAM62B | Hs.649908 | | X | | | | | | X | | | X | |
| similarity 62 member B | | Day 7 | | | | | | | | | | | | |
| Transcribed locus | | Hs.42650 | | | | | | | | | X | X | | |
| | | Day 7 | | | | | | | | | | | | |

| | | | | | | | | | | | | |
|---|---|---|---|---|---|---|---|---|---|---|---|---|
| Accuracy of 10-fold cross- | 93 | 93 | 93 | 93 | 93 | 93 | 93 | 93 | 90 | 90 | 100 | 100 |
| validation (%) | | | | | | | | | | | | |
| Accuracy of 1-fold blind | 80 | 80 | 80 | 80 | 80 | 90 | 90 | 90 | 87 | 87 | 80 | 73 |
| prediction (%) | | | | | | | | | | | | |
| Accuracy of 10-fold blind | 81 | 80 | 81 | 80 | 81 | 85 | 85 | 88 | 84 | 84 | 76 | 72 |
| prediction (%) | | | | | | | | | | | | |

| | | | DAMIP model predictive signatures | | | | | | | | | |
|---|---|---|---|---|---|---|---|---|---|---|---|---|
| | Gene | | Train on trial 2, test on trial 1 | | | | | | | | | |
| Gene name | symbol | Gene ID | 5 | 6 | 7 | 8 | 9 | 10 | 11 | 12 | 13 | 14 |
| Solute carrier | SLC2A6 | Hs.244378 | X | | X | | | X | X | X | X | |
| family 2 | | Day 7 | | | | | | | | | | |
| (facilitated glucose | | | | | | | | | | | | |
| transporter), member 6 | | | | | | | | | | | | |
| Eukaryotic translation | EIF2AK4 | Hs.412102 | | | | X | | X | | | | X |
| initiation factor 2 alpha | | Day 7 | | | | | | | | | | |
| kinase 4 | | | | | | | | | | | | |
| Integrin, alpha L (antigen | ITGAL/LFA-1 | Hs.174103 | | X | X | X | X | | | | | |
| CD11A) | | Day 7 | | | | | | | | | | |
| C-terminal binding | CTBP1 | Hs.208597 | | X | | | | | | | | |
| protein 1 | | Day 7 | | | | | | | | | | |
| Tyrosine 3- | YWHAE | Hs.513851 | | | | | X | | | X | | |
| monooxygenase/tryptophan | | Day 3 | | | | | | | | | | |
| 5-monooxygenase | | | | | | | | | | | | |
| activation protein | | | | | | | | | | | | |
| Transcribed locus | | Hs.619443 | X | X | X | X | X | X | X | X | X | X |
| | | Day 7 | | | | | | | | | | |
| Protein phosphatase 1, | PPP1R14A | Hs.631569 | | | | | | | | | | X |
| regulatory (inhibitor) | | Day 3 | | | | | | | | | | |
| subunit 14A | | | | | | | | | | | | |
| Family with sequence | FAM62B | Hs.649908 | | | | | | | | | X | |
| similarity 62 member B | | Day 7 | | | | | | | | | | |
| Transcribed locus | | Hs.42650 | | | | | | | X | | | |
| | | Day 7 | | | | | | | | | | |

| | | | | | | | | | | |
|---|---|---|---|---|---|---|---|---|---|---|
| Accuracy of 10-fold cross- | 100 | 100 | 90 | 90 | 90 | 90 | 90 | 90 | 100 | 100 |
| validation (%) | | | | | | | | | | |
| Accuracy of 1-fold blind | 73 | 73 | 73 | 73 | 73 | 73 | 87 | 73 | 80 | 73 |
| prediction (%) | | | | | | | | | | |
| Accuracy of 10-fold blind | 75 | 71 | 73 | 71 | 71 | 75 | 84 | 73 | 76 | 70 |
| prediction (%) | | | | | | | | | | |

This table summarizes the classification rules that have tenfold cross-validation prediction of at least 80%. Tenfold cross validation on trial 1 resulted in eight different DAMIP predictive signatures, each of which had a tenfold unbiased estimate of 93% prediction rate in trial 1. SLC2A6 and EIF2AK4 are represented in several signatures. Blind prediction of rules developed from trial 1 on trial 2 data produced prediction accuracies in the 80-90% range. Tenfold blind predictions were also carried out to evaluate the consistency of the classification rules obtained by subsets of training data only. Here, for trial 1, rule 1 in the tenfold blind test, nine of the ten resulting rules resulted in 80% correct prediction on the blind data from trial 2, and one rule resulted in 90% correct prediction. Thus, the average unbiased prediction rate on the blind data was 81%. However, when the classification rules were generated using the entire training set, they predicted the blind data with an accuracy of 80% singlefold blind test). Conversely, 14 different discriminatory predictive signatures were obtained when trial 2 was used as the training set, with unbiased classification rates in the range 90-100%. EIF2AK4 and SLC2A6 were also represented in these models. Blind prediction on independent trial 1 yielded 73-87% prediction accuracy. Although gene expression data across various time points were all input into the predictive model, most of the discriminatory signature sets (16 out of 22) consisted of only the day 7 expression relative to the day 0.

Many of the genes contained in the DAMIP and ClaNC signatures were verifiable using RT-PCR (Table 7). Although gene expression data across various time points were all input into the predictive model, most of the discriminatory signature sets consisted of only day 7 expression relative to day 0, Specifically, among the 22 rules (Table 6), only 6 rules involved signature sets that include different time measurements (day 3). Notably, signature sets were identified that provided at least 87% of prediction accuracy (Table 6). Although it may be convenient to select the best rules on the basis of the best prediction accuracy for future biological investigation, premature elimination of those results that offer 70% prediction rate should be caution against, as some of the most commonly used diagnostic tests, such as the Pap smear, produce similar prediction rates.

TABLE 7
RT-PCR confirmation of 15 genes used in CD8⁺ T cell activation prediction models

| | | | | | Pearson | |
|---|---|---|---|---|---|---|
| Symbol | UniGene | TaqMan assay | Day | Model | r | P-value |
| RGS1 | Hs.75256 | Hs0017526_m1 | 3 | ClaNC | 0.8924 | 0.0005 |
| CD69 | Hs.208854 | Hs0015399_m1 | 3 | ClaNC | 0.8837 | 0.0007 |
| ALDH3B1 | Hs.523841 | Hs00997594_m1 | 3 | ClaNC | 0.8117 | 0.0002 |
| CXCR7 | Hs.471751 | Hs00664172_s1 | 3 | ClaNC | 0.788 | 0.0068 |
| C1QB | Hs.8986 | Hs00608019_m1 | 3 | ClaNC | 0.7803 | 0.0077 |
| ASGR2 | Hs.16247 | Hs00154160_m1 | 7 | ClaNC | 0.7202 | 0.0025 |
| JUN | Hs.525704 | Hs99999141_s1 | 3 | ClaNC | 0.7184 | 0.0193 |
| CXCR7 | Hs.471751 | Hs00664172_s1 | 7 | ClaNC | 0.7078 | 0.0032 |
| ATP6V1E1 | Hs.517338 | Hs00762211 S1 | 3 | ClaNC | 0.6841 | 0.0049 |
| ASGR2 | Hs.16247 | Hs00154160 m1 | 3 | ClaNC | 0.6056 | 0.0167 |
| SLC2A6 | Hs.244378 | Hs00214042_m1 | 7 | ClaNC, DAMIP | 0.5494 | 0.0339 |
| MEF2A | Hs.268675 | Hs00271535_m1 | 7 | ClaNC | 0.5423 | 0.0368 |
| CTBP1 | Hs.208597 | Hs00179922_m1 | 7 | DAMIP | 0.4634 | 0.0819 |
| ITGAL | Hs.174103 | Hs00158238_m1 | 7 | DAMIP | 0.4517 | 0.091 |
| EIF2AK4 | Hs.412102 | Hs00383836_m1 | 7 | ClaNC, DAMIP | 0.4124 | 0.1266 |

The Pearson r is calculated for the log₂-fold change microarray data versus the relative RT-PCR measurements on either Day 3/Day 0 or Day 7/Day 0 with data points from each of the subjects samples assayed.

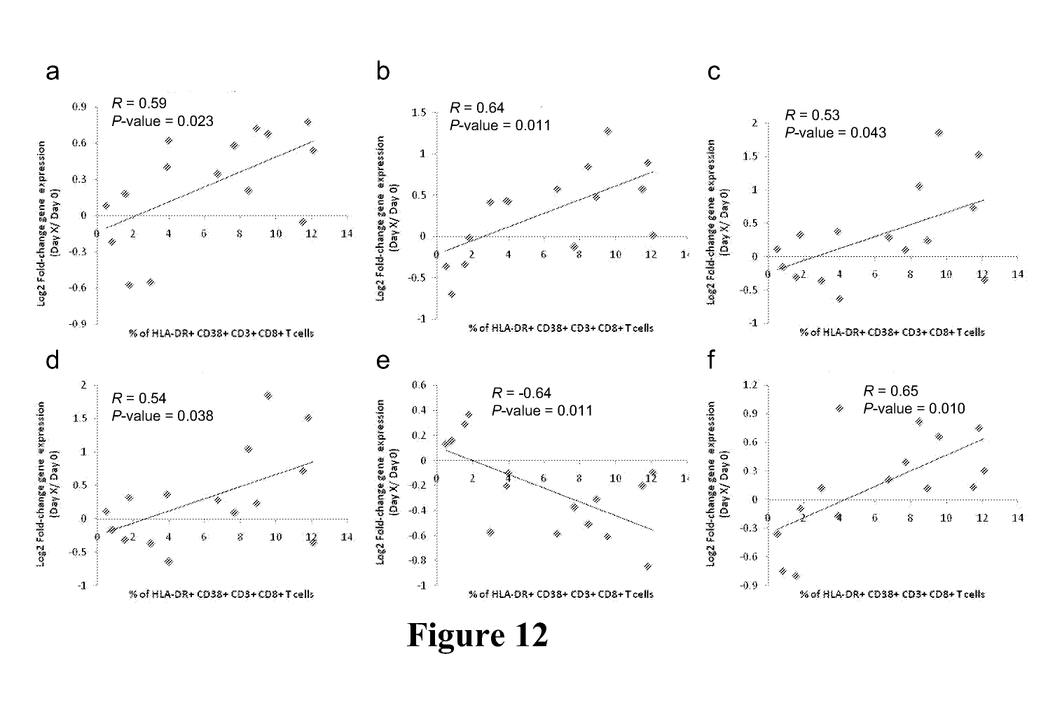

Finally, the repeated representation of EIF2AK on multiple DAMIP model signatures and in the ClaNC model raised the possibility that this gene has a key function in mediating CD8+ T cell responses to YF-17D. EIF2AK4 (also called GCN2 (mammalian general control nonderepressible 2)) serves a function in the so-called ‘integrated stress response’ by regulating translation in response to various stress signals from the environment. It does so by phosphorylating the α-subunit of translation initiation factor 2 (eIF2α), which results in the shutdown of translation of most proteins in the cell. In contrast, the expression of proteins responsible for damage repair is increased by a process that involves redirection of these mRNAs from polysomes to discrete cytoplasmic foci known as ‘stress granules’ for transient storage 20. Consistent with that, YF-17D induced phosphorylation of eIF2α (FIG. 11a) and the formation of stress granules (FIG. 11b). Moreover, several other genes encoding molecules involved in the stress-response pathway, including calreticulin, protein disulfide isomerase, the glucocorticoid receptor and c-Jun, were upregulated in response to YF-17D, and this correlated with the CD8+ T cell response (FIG. 12).

(4) Signatures that Predict Antibody Responses

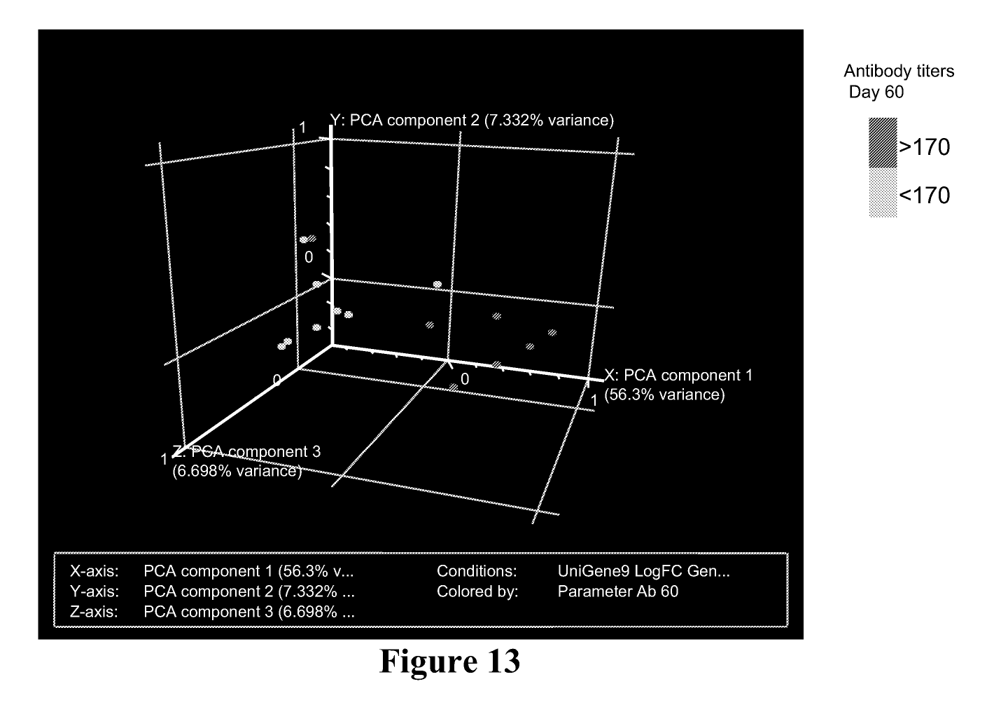

To further strengthen the DAMIP results, predictions were carried out on the B cell antibody responses (Table 8). For the B cell analysis, the goal was to identify an early gene signature that correlated with the magnitude of the neutralizing antibody response in the 15 individuals in the first trial. Here, trial 1 consisted of six subjects in the high group and nine in the low group, and trial 2 consisted of four subjects in the high group and six in the low group (FIG. 13). Genes that correlated with the magnitude of the neutralizing antibody response at day 60 were identified as was done for CD8+ T cells (described above and in Methods). To visualize how well the genes identified by the relative expression and P-value cutoffs sorted the subjects in terms of the antibody responses, unsupervised principal component analysis was performed. The genes segregated the subjects into two subgroups with a neutralizing antibody titer cutoff of 170 (FIG. 13). Next, the DAMIP model was applied to determine gene signatures that could predict the antibody response in trial 2. In trial 2, because antibody titers at day 60 were not available, the titers at day 90 were used. As before (Table 6), those results were summarized with tenfold cross-validation scores of at least 80%. Here, whereas the classification rules from trial 1 uniformly predicted all the trial 2 cases correctly (resulting in singlefold blind prediction of 100%), the rules developed using trial 2 resulted in at most 80% singlefold blind prediction accuracy (Table 8). TNFRSF17, a receptor for the B cell growth factor BLyS-BAFF; A000383), was present in all the predictive signature sets of the DAMIP model, and several genes, including KBTBD7 and BEND4, appeared in multiple signature sets (Table 8). Notably, many of these genes were verified using RT-PCR (Table 9). These two independent analyses of T cells and B cell responses confirmed that the DAMIP method is suitable for identifying predictive signature sets. For both T cell and B cell analysis, the classification rules generated from trial 1 provided higher blind prediction accuracy for trial 2 data than did the reverse analysis. This may be partly because trial 1 consisted of a slightly larger sample size.

TABLE 8
Genomic signatures that predict the magnitude of the neutralizing antibody responses using the DAMIP model

| | | | DAMIP model predictive signatures | | | | | | | | | | | | | | |
|---|---|---|---|---|---|---|---|---|---|---|---|---|---|---|---|---|---|
| | Gene | | Train on trial 1, test on trial 2 | | | | | | | | | | Train on trial 1, test on trial 2 | | | | |
| Gene name | symbol | Gene ID | 1 | 2 | 3 | 4 | 5 | 6 | 7 | 8 | 9 | 10 | 1 | 2 | 3 | 4 | 5 |
| BEN domain- | BEND4 | Hs.120591 | | | | X | X | X | X | X | | X | X | | | X | |
| containing 4 | | | | | | | | | | | | | | | | | |
| Transcribed | | Hs.139006 | X | | | | | X | | | X | X | | | | | |
| locus | | | | | | | | | | | | | | | | | |
| 6-Phosphofructo- | PFKFB3 | Hs.195471 | | X | | | | | | | | | | | | | |
| 2-kinase/fructose- | | | | | | | | | | | | | | | | | |
| 2,6-biphosphatase 3 | | | | | | | | | | | | | | | | | |
| Tumor necrosis | TNFRSF17 | Hs.2556 | X | X | X | X | X | X | X | X | X | X | X | X | X | X | X |
| factor receptor | | | | | | | | | | | | | | | | | |
| superfamily, | | | | | | | | | | | | | | | | | |
| member 17 | | | | | | | | | | | | | | | | | |
| Tumor protein | TPD52 | Hs.368433 | | | | X | | | X | | X | | | X | | X | |
| D52 | | | | | | | | | | | | | | | | | |
| Transcribed | | Hs.481166 | | | | | X | X | | X | | X | | | | | |
| locus | | | | | | | | | | | | | | | | | |
| Kelch repeat | KBTBD7 | Hs.63841 | X | X | X | X | X | X | X | X | X | X | | | X | | X |
| and BTB | | | | | | | | | | | | | | | | | |
| (POZ) domain | | | | | | | | | | | | | | | | | |
| containing 7 | | | | | | | | | | | | | | | | | |
| Transcribed | | Hs.649726 | | | | | | | X | X | X | | | | | | X |
| locus | | | | | | | | | | | | | | | | | |
| Nucleosome | NAP1L2 | Hs.66180 | | | X | | | | | | | X | | | | | |
| assembly | | | | | | | | | | | | | | | | | |
| protein 1-like 2 | | | | | | | | | | | | | | | | | |

| | | | | | | | | | | | | | | | |
|---|---|---|---|---|---|---|---|---|---|---|---|---|---|---|---|
| Accuracy of 10-fold cross- | 80 | 80 | 80 | 87 | 87 | 80 | 80 | 80 | 80 | 80 | 89 | 89 | 89 | 89 | 89 |
| validation (%) | | | | | | | | | | | | | | | |
| Accuracy of 1-fold blind | 100 | 100 | 100 | 100 | 100 | 100 | 100 | 100 | 100 | 100 | 73 | 73 | 73 | 73 | 80 |
| prediction (%) | | | | | | | | | | | | | | | |
| Accuracy of 10-fold blind | 97 | 99 | 94 | 92 | 96 | 98 | 92 | 93 | 93 | 94 | 72 | 71 | 75 | 70 | 79 |
| prediction (%) | | | | | | | | | | | | | | | |

Analysis of signatures that predict the neutralizing antibody responses. Here all the discriminatory predictive signature sets turned out to consist of day 7 gene expression only. Further, training on trial 1 produces a high blind prediction accuracy on trial 2. TNFRSF17 was present in all the predictive signature sets of the DAMIP model, and several genes, including KBTBD7 and BEND4 appeared in several signature sets.

TABLE 9
RT-PCR validation of genes in the DAMIP models for signatures
that predict neutralizing antibody titers

| Symbol | UniGene | Day | Pearson r | P-value |
|---|---|---|---|---|
| BEND4 | Hs.120591 | 7 | 0.764 | 0.00002 |
| KBTBD7 | Hs.63841 | 7 | 0.543 | 0.02510 |
| TNFRSF17 | Hs.2556 | 7 | 0.784 | 0.000001 |
| TPD52 | Hs.368433 | 7 | 0.530 | 0.00667 |

(5) Identifying Genes Induced by YF-17D in Most Vaccinees.

The raw Affymetrix microarray probe data was assembled into probe sets representing individual genes based on the updated UniGene Build 199, Jan. 16, 2007 to yield a list of 20,078 genes based on a previously published method instead of using Affymetrix predefined probe sets. R was used to assemble the probe sets in combination with RMA pre-processing, which includes global background adjustment and quantile normalization. Values below a minimum threshold of normalized fold change in expression of 0.01 for microarrays were reset to that threshold. Gene expression at time points post-vaccination were converted to fold changes by subtracting the pre-vaccination day 0 expression value. Genes with fold change in expression patterns that were similar among most subjects within a trial over time, were detected by identifying genes with normalized Log 2 transformed fold change gene expression values >0.5 or <0.5 in >60% of the subjects, at days 3 or 7 and then tested for statistical significance by ANOVA adjusted with the Benjamini and Hochberg False Discovery Rate method with a cutoff of 0.05 in Genespring (Agilent Technologies).

One-way ANOVA was used to test for differences on the expression levels of each gene among days 0, 3 and 7. This test does not depend on the number of genes since it is run independently for each gene. Benjamini and Hochberg False Discovery Rate method depends on the number of tests performed and a pre-selection filter may affect the multiple testing corrections. However, the pre-filtering cut-off used was very low (only a Log 2 transformed fold change gene expression values of 0.5 or 41% increase or decrease on the gene expression levels in at least ⅗ of subjects) and necessary only to remove genes that did not fluctuate with time, which are often unexpressed/low expressed genes. Therefore, the pre-selection filter did not compromise the findings. Nevertheless, a testing of the whole dataset by ANOVA was explored without any pre-selection filter. This resulted in a list of 22 genes (Table 1). This low number of genes is absolutely expected. A gene list with 20,000 genes will require for the gene with lowest P-value given by ANOVA an adjusted P-value lower than 0.0000025 (0.05/20,000) and for the gene with the second lowest P-value, an adjusted P-value lower than 0.000005 (0.05/(20,000/2)). However there was still a question as to whether this selection criterion were too stringent since it was not detecting the increased transcription of CD38 and IP-10, which was known to be increased by flow cytometry and ELISA, data respectively (FIG. 8 & FIG. 1). Prefiltering allowed the detection of these genes that had already been “verified” at the protein level and identified an additional subset a genes with close biological interactions with the 22 genes already selected (Table 1). Furthermore, this expanded list of genes indicated a role for complement, which was verified by ELISA (FIG. 5), and also had many more genes that can be verified by RTPCR (Table 1). Therefore while it is likely that omitting the prefiltering step may result in a more rigorous statistical analysis, said analysis may be too stringent and exclude potentially biologically relevant genes.

(6) Identifying Genes that Correlate with Magnitudes of Immune Responses.

Genes, whose expression correlated with the magnitude of the T-cell responses were identified by comparing the % of CD38+ HLA-DR+ (activated) CD8+ T-cells to the normalized Log 2 transformed gene expression values. Genes with >25% of the subjects having >0.5 or <0.5 change were analyzed by the Linear Model (lm) function in R to identify genes with a slope P-value <0.05. A predictive model of T cell responses was generated using ClaNC run within R. Principle Component Analysis (PCA) to visually reduce and summarize gene expression variance among the subjects was conducted in Genespring. The student t-test was performed in Prism to test whether genes displayed a significant difference between subjects when they were grouped by T cell responses. Gene networks and functional relationships were analyzed with Ingenuity Pathways Analysis (Ingenuity Systems) and the DAVID Bioinformatics Database. Transcription factor binding sites of gene lists were analyzed in TOUCAN v3.0.2 using the TRANSFAC v7.0 database of eukaryotic transcription factors. Binding site motifs were scanned for in the DNA sequence 2000 bases upstream through 200 bases downstream flanking the first exon of each gene with a double prior of 0.1 and the genomic background noise model based on the third order Markov Model from the Human Eukaryotic Promoter Database (Human EPD 3). RT-PCR genes from the Applied Biosystems Custom TaqMan Gene Expression plate, was normalized to the average Ct value of the housekeeping genes 18S rRNA (Hs99999901_s1), ACTB (Hs99999903_m1), and B2M (Hs99999907_m1) and then the difference in normalized Ct value between day 3 and 7 versus day 0 was calculated. Correlation between the fold changes in microarray and RT-PCR data were calculated using Prism. Genes believed to change with time post vaccination were tested for statistical significance by ANOVA adjusted False Detection Rate method with a cutoff of 0.05 in Genespring, as with the microarray data. For the CD8 predictive model, correlation between microarray and RTPCR data for each individual gene was analyzed using Prism. For analysis of data from the experiment of stimulating PBMCs with YF-17D, genes were selected that were up or down regulated by a factor of 0.5 fold in the Log 2 scale, after either 3 or 12 hours of stimulation with YF-17D, compared to cells cultured in media alone. The student t-test was performed for comparing YF-17D to media alone at 3 and 12 hours. The genes commonly modulated in both independent trials were analyzed for statistically overrepresented transcription factor binding sites in TOUCAN v3.0.2 using the TRANSFAC v7.0 public database of eukaryotic transcription factors.

(7) Discriminant Analysis Via Mixed Integer Programming.

There are five fundamental steps in discriminant analysis: (i) determine the data for input and the predictive output classes; (ii) gather a training set of data (including output class) from human experts or from laboratory experiments. Each element in the training set is an entity with corresponding known output class; (iii) determine the input attributes to represent each entity; (iv) identify discriminatory attributes and develop the predictive rules; (v) validate the performance of the predictive rules.

Utilizing the technology of large-scale discrete optimization and support-vector machines, novel predictive models DAMIP, were developed that simultaneously include the following features: the ability to classify any number of distinct groups; the ability to incorporate heterogeneous types of attributes as input; a high-dimensional data transformation that eliminates noise and errors in biological data; constraints to limit the rate of misclassification, and a reserved judgment region that provides a safeguard against over-training (which tends to lead to high misclassification rates from the resulting predictive rule); and successive multi-stage classification capability to handle data points placed in the reserved judgment region.

In the analysis, each Trial forms a data set. The entity in the dataset is an individual vaccinee, and the measurable attributes for each entity consists of the time measurement of gene array data described in the data collection part. For the T cell analysis, the group in each Trial is determined by the magnitude of the CD8+ T cell response. There are about 800 total measurable gene attributes (of mixed time points) for each entity. Disclosed herein are 2 groups of vaccinees in the T cell analysis (“high” group and “low” group). Each experiment consists of the following two parts: a) Develop a classification rule using a training dataset (Trial 1), b) Use the rule developed from the training set to predict the group status of independent unknown entities (from Trial 2). The experiment is then repeated using Trial 2 as the training set and Trial 1 for blind prediction. For the B cell analysis, the group in each Trial is determined by the magnitude of the neutralizing antibody titers. Again there are the “high” and “low” groups. There are about 1600 total measurable gene attributes (of mixed time points) for each entity.

Performance and validation of the rules is reported in: (a) 10-fold cross validation which reports the unbiased estimate of classification correctness in the training stage, and (b) in 1-fold and 10-fold blind prediction of the independent Trial entities which report the prediction accuracy of new and unknown data. While 10-fold cross validation offers the confidence interval and reliability of the rules generated and tested within the same Trial of patients, the blind test provides a further measurement of its practical usage across different independent Trials.

(8) 10-Fold Cross-Validation

To obtain an unbiased estimate of the reliability and quality of the derived classification rules, ten-fold cross validation is performed. In the ten-fold cross validation procedure, the training set is randomly partitioned into ten subsets of roughly equal size. Ten computational experiments are then run, each of which involves a distinct training set made up of nine of the ten subsets and a test set made up of the remaining subset. The classification rule obtained via a given training set is applied to each point in the associated test set to determine to which group the rule allocates it. The process is repeated until each subset has been used once for testing. The cumulative measure of correct classification of the ten experiments provides the unbiased estimation of correct classification.

(9) 1-Fold Blind Predictions

In 1-fold blind prediction, a classification rule is first developed using all the training data. This rule is then applied to each entity in the blind data to predict its group status. The percent of correct prediction of the blind data entities is recorded, providing a measure of overall prediction accuracy.

(10) 10-Fold Blind Predictions

In 10-fold blind predictions, 10 classification rules as in 10-fold cross-validation were generated each rule is generated using nine of the ten subsets of the training sets. Then, the blind data (all of them) are tested on this rule. This process is repeated ten times, and the average cumulative prediction forms the unbiased prediction correctness of the blind data. To develop the classification rule from a given training set, the training data is fed into the DAMIP model. The feature selection algorithm inside the model determines, out of the large set of gene measurements, a subset of genes a discriminatory signature that may help to classify entities in the training set into the two groups. The classification rate associated with the signature set (obtained by performing ten fold cross validation using the selected signature features) is then recorded. This “learning” process is repeated, each time an updated discriminatory signature set and associated classification rate are obtained and recoded. Users can pre-set the number of discriminatory gene measurements that are desired in each signature set. Since the number of patients in each clinical Trial is rather small, each signature set was set to contain at most 5 gene attributes. Users can also pre-set an appropriate target value for the classification rate. Thus the machine continues to learn (generate signatures sets and associated classification rates) and terminate when the target classification rate is achieved, or when it reaches a level of correct classification and cannot improve any further (in this case, it may not have achieved the pre-set target rate). In the study, the learning process was terminated when the resulting classification rate reached 80%. Developing a classification rule is computationally expensive due to the combinatorial nature of the feature selection process. However, once a rule has been obtained, it is easy and inexpensive to apply it to new unknown entities to predict group membership.

To perform a blind test, simply input each entity from an independent Trial and process it through the classification rule (obtained from a training set). This takes less than a second of CPU time.

In Brooks and Lee, 200817, it was proved the classification rule resulted from DAMIP is strongly universally consistent. It consistently results in low inter-group misclassification rates; it is insensitive to the specification of prior probabilities, yet capable of reducing misclassification rates when the number of training observations from each group is different. Further, the DAMIP rule is proved to be stable regardless of the proportion of training observations from each group.

With regards to why Trial 1->Trial 2 DAMIP predictions were significantly more successful for the antibody titer predictions that the Trial 2>Trial 1, since As Trial 2 is smaller than Trial 1, one possible explanation for the discrepancies in predictive power is that the ranges of individual variability in genetic responses is more completely captured in Trial 1 than Trial 2. In other words, out of the ranges of responses that humans can make, Trial 2 may contain a subset of those found in Trial 1. Therefore while Trial 1 based models only need to interpolate predictions for Trial 2, Trial 2 based models may need to extrapolate predictions for some of the Trial 1 subjects. “Interpolation” and “extrapolation” are traditionally thought of in terms of polynomial functions, in which case extrapolating data is associated with greater uncertainty and greater likelihood of inaccurate prediction.

b) Discussion

Here, an interdisciplinary approach was adopted using multiplex cytokine analysis, flow cytometry and microarray transcriptional profiling to characterize signatures of YF-17D vaccine responses. Because the high numbers of genes in microarray analysis increase the likelihood of false positives, the observed transcriptional profiles were verified with a second independent study using different subjects vaccinated a year later with a new vaccine lot. The results indicated that several innate immune mechanisms are induced by YF-17D and that some signatures can be used to predict the strength of the adaptive immune response.

Of the 24 cytokines assayed, IP-10 and IL-1α were significantly induced after vaccination. This is consistent with similar results obtained during other flavivirus infections, such as dengue, West Nile virus and tick-borne encephalitis. Thus, IP-10 and IL1A (IL-1α) are reliable markers of YF-17D vaccination, and they can play an integral role in responses to other flaviviruses. A comprehensive microarray analysis was performed to identify genomic signatures that correlated with the immune response. This analysis revealed molecular events observed in innate immune control of viruses. In particular, molecules involved in innate sensing of viruses, such as TLR7, cytoplasmic receptors of 2,5′-OAS family members 1, 2, 3 and L, RIG-I, and MDA-5, as well as transcription factors that regulate type I interferons (IRF7, STAT1), were induced; consistent with this, YF-17D was also shown to signal through RIG-I and MDA-5. In addition, the upregulation of ISG15 and of HERC5 and UBE2L6, which participate in ISGylation was also detected. The four upregulated genes that are involved in ubiquitination may also be recruited into the ISGylation pathway, or they may remain as part of the ubiquitin pathway, where they form part of a negative feedback loop to downregulate the abundance of specific proteins. Furthermore, there was also upregulation of LGP2, which negatively regulates the response mediated by RIG-I and MDA-5. Thus, YF-17D vaccination induced a gene signature characteristic of viral infections; however there was no correlation between the induction of such genes and the magnitude of the CD8 T+ cell response.

A different signature was successful in predicting the CD8+ T cell response. C1QB was a key positive predictor of T cells in the ClaNC model; this is consistent with the upregulation of C3AR1 and C1IN and increased plasma C3a concentrations. Consistent with this, deficiencies in C1q, C3, C4, factor B, factor D, CR1 and CR2 each individually increase mortality, and diminish T cell and antibody responses, against the closely related flavivirus West Nile in mice. In addition, two factors, SLC2A6 (GLUT1) and EIF2AK4, were present in the predictive signatures identified using two independent classification models. SLC2A6 belongs to a family of membrane proteins that regulate glucose transport and glycolysis in mammalian cells. Notably, in the signature derived in the ClaNC model, several other family members, SLC16A5, SLC25A13, SLC39A11, were also represented, indicating a possible role for glucose metabolism in regulating the CD8+ T cell response. Although the putative roles of such proteins in regulating immunity is not yet known, recent work suggests that, in T cells, CD28 signaling regulates glucose metabolism through expression of GLUT1. EIF2AK4 (also known as mammalian general control non-derepressible-2 (GCN2)) regulates protein synthesis in response to environmental stresses by phosphorylating the α-subunit of initiation factor 2 (eIF2α). In this stress response, the expression of proteins responsible for damage repair is increased, whereas translation of constitutively expressed proteins is aborted by redirection of these mRNAs from polysomes to discrete cytoplasmic foci known as stress granules for transient storage. Consistent with this, YF-17D induced the phosphorylation of eIF2α and formation of stress granules. Moreover, several other genes involved in the stress response pathway, including calreticulin, protein disulfide isomerase and the glucocorticoid receptor JUN, were modulated in response to YF-17D and correlated with the CD8+ T cell response. Recent work has shown an antiviral effect of EIF2AK4 against RNA viruses, but the consequence of this for adaptive immunity is not known. It is thus tempting to speculate that the induction of the integrated stress response in the innate immune system might regulate the adaptive immune response to YF-17D, and perhaps other vaccines or microbial stimuli. Finally, in the case of antibody responses, the gene for TNFRSF17, a receptor for the B cell growth factor BLyS-BAFF23, was key in the predictive signatures of the DAMIP model. Notably, BLyS-BAFF is thought to optimize B cell responses to B cell receptor- and TLR-dependent signaling.

YF-17D is highly efficacious, since epidemiological studies indicate that this vaccine confers protection in 80-90% of vaccinees 3; the mechanism of protection is believed to be neutralizing antibodies, although cytotoxic T cells are also believed to play a role. This study uses YF-17D simply as a model to provide methodological evidence that critical parameters of protective immunity (that is, CD8+ T cell and antibody responses) can indeed be predicted early after vaccination. The identification of gene signatures that correlate with, and are capable of predicting, the magnitudes of the antigen-specific CD8+ T cell and neutralizing antibody responses provides the first methodological evidence that vaccine-induced immune responses can indeed be predicted. This in turn indicates that such approaches can predict the immunogenicity and/or protective efficacy of emerging vaccines.

In summary, systems biology approaches not only permit the observation of a global picture of vaccine-induced innate immune responses but can also be used to predict the magnitude of the subsequent adaptive immune response and uncover new correlates of vaccine efficacy. Using two independent trials, the DAMIP method was found to be useful in determining these correlates. This argument is further strengthened by examining independently both T cell and B cell responses using the DAMIP method. Further application of such approaches are of interest to vaccine development in several ways. For example, different comparisons, such as vaccine responders versus vaccine nonresponders or good versus poor vaccines, can help to identify possible innate correlates of protection, previously unrecognized mechanisms of vaccine action, and early screening strategies of multiple vaccine candidates, hence facilitating research and development efforts. The recent setback with the Merck HIV vaccine 35 underscores the imperative for such approaches in predicting the immunogenicity and protective capacity of vaccines.

c) Methods

(1) Clinical Study Organization.

The research was approved by the Emory University Institutional Review Board. Enrolled volunteers were healthy, aged 18 to 45, and signed a written informed consent form. Potential volunteers were excluded from participating in the study if they were pregnant or if they had been vaccinated previously with YF-17D. Blood samples for multiplex analysis of cytokines, innate immune cell and microarray analysis were collected in citrate-buffered cell preparation tubes (CPTs; Vacutainer; BD) at days 0, 1, 3, 7 and 21 after vaccination. PBMCs were frozen in DMSO with 10% FBS and stored at −80° C. For T cell and antibody assays, blood was collected in citrate-buffered CPTs on days 0, 15 and 60. The tubes of blood were processed according to the manufacturer's protocol.

(2) Multiplex Analysis.

Plasma samples from CPTs were stored at −80° C. before cytokine analysis. Assays were performed with the Beadlyte Human 22-Plex Multi-Cytokine Detection System with the addition of interferon-α2 and IL-1 receptor-α Beadmates to make a 24-plex assay (Upstate). Samples were run in duplicate following the manufacturer's protocol on a Bio-Plex Luminex-100 station (Bio-Rad). Data were normalized using the prevaccination cytokine level (that is, log 2Cd-log 2C0, where Cd is the cytokine concentration on day d). The data were tested for significance in Prism by one-way ANOVA followed by the Tukey post hoc test.

(3) Flow Cytometric Analysis.

PBMCs from all time points for an individual were thawed, stained and acquired in parallel. Monocytes were gated as HLA-DR+CD14+ with the addition of CD16 to delineate the subpopulation of inflammatory monocytes. Myeloid DCs were gated as lineage cocktail HLA-DR+CD11c+, and plasmacytoid DCs were gated as lineage cocktail HLA-DR+CD123+. CD86 expression was used to indicate the percentage of activated antigen-presenting cells within each population. The log 2-transformed values for the percentages of CD86+ cells were normalized relative to baseline values. For T cell activation, after gating on the CD8+CD3+ T cells, the percentage of CD38+HLA-DR+ cells was calculated. Antibodies were obtained from BD Biosciences (HLA-DR, 340690; lineage cocktail, 340546; CD11c, 559877; CD14, 555399; CD123, 340545; CD86, 555658). The data were tested for significance in Prism by one-way ANOVA followed by the Tukey post hoc test.

(4) Assay for Yellow Fever Virus (YFV) Neutralizing Antibodies.

Serum or plasma samples were heated to 56° C. for 30 min to inactivate complement. YFV neutralizing antibodies were measured by cytopathic effect (CPE) (trial 1) or by plaque reduction neutralization test (PRNT) (trial 2). In brief, for neutralizing antibodies by CPE, plasma dilutions in triplicate were incubated with 1,000 plaque-forming units of YFV at 37° C. for 1 h in 96-well flat-bottomed plates. Five thousand Vero cells were added to each well and the plates stained with crystal violet after 4 d. The last dilution that showed an intact monolayer of Vero cells with no CPE was used as the antibody titer. For the PRNT, various dilutions of the sera were incubated overnight at 4° C. with 200 plaque-forming units of YFV. Vero cell monolayers in drained six-well plates were incubated with this virus-serum mixture for 1 h at 37° C. The wells were overlaid with a mix of agarose and 2XM199 medium and plaques counted 3-4 d later using neutral red. Because the CPE and PRNT assays have different scales of neutralizing antibody titers, the results between the two trials were normalized by their medians; that is, normalized subject X value in trial 2=(trial 1 median/trial 2 median)×subject X value in trial 2.

(5) RNA Isolation and Microarray and RT-PCR Data Generation.

After PMBC isolation from CPTs, 2×106 cells were lysed in 1 ml of TRIzol (Invitrogen) and stored at −80° C. After all time points were collected for a subject, the samples were thawed, and the RNA isolation proceeded according to the manufacturer's protocol. Total RNA sample quality was evaluated by spectrophotometer to determine quantity, protein contamination and organic solvent contamination, and an Agilent 2100 Bioanalyzer was used to check for RNA degradation. Two-round in vitro transcription amplification and labeling was performed starting with 50 ng intact, uncontaminated total RNA per sample, following the Affymetrix protocol. After hybridization on Human U133 Plus 2.0 Arrays for 16 h at 45° C. and 60 r.p.m. in a Hybridization Oven 640 (Affymetrix), slides were washed and stained with a Fluidics Station 450 (Affymetrix). Scanning was performed on a seventh-generation GeneChip Scanner 3000 (Affymetrix), and Affymetrix GCOS software was used to perform image analysis and generate raw intensity data. Initial data quality was assessed by background level, 3′ labeling bias, and pairwise correlation among samples. For this analysis, Affymetrix Human Genome U133 Plus 2.0 Array was used, but instead of using Affymetrix's sequence clusters to define genes, which is based on the UniGene database build 133, 20 Apr. 2001, gene sequence clusters were based on the updated UniGene build 199, 16 Jan. 2007, to yield a list of 20,078 genes. For RT-PCR analysis, Applied Biosystems constructed a custom TaqMan Gene Expression Plate Assay for 48 genes in their database. Two-step RT-PCR was performed. Values obtained by RT-PCR of genes from the custom TaqMan Gene Expression plate (Applied Biosystems) were normalized to the average cycling threshold value of the ‘housekeeping’ genes encoding 18S rRNA (Hs99999901_s1), β-actin (Hs99999903_m1) and 132-microglobulin (Hs99999907_m1), and then the difference in normalized cycling threshold values between days 3 and 7 versus day 0 was calculated. Significance was determined by one-way analysis of variance over days 0, 3, and 7.

(6) In Vitro Stimulation of Human PBMCs with YF-17D.

PBMCs from two healthy, unvaccinated donors were isolated and plated at 1×106 cells per well in 48-well plates with 1 ml RPMI with 10% FBS and penicillin plus streptomycin. The cells were cultured in the presence or absence of YF-17D at a multiplicity of infection of 1. After 3 and 12 h, RNA was isolated from the cells and processed for microarray analysis. For these experiments, the Affymetrix Human Genome 133A 2.0 Array was used. This microarray contains a subset of genes found on the Human 133 Plus 2.0 Array, which was used in the analysis of the vaccinees. The analysis was performed as described in the Supplementary Methods.

(7) Data Analysis.

Full details are in Supplementary Methods. Immunofluorescence, immunoblot analysis and ELISA. BHK cells were cultured on cover slips in 24-well plate and stimulated with YF-17D. Cells were fixed with 3.7% formaldehyde and permeabilized with 0.5% saponin (Sigma). Cells were then incubated with anti-TIAR (C-18) (Santa Cruz 1749, 1:50) for 2 h at room temperature. After washing, cells were incubated with donkey anti-goat secondary antibody coupled to fluorescein isothiocyanate (Santa Cruz 2024, 1:100). F-actin structure was visualized using BODIPY 558/568 phalloidin (Invitrogen) and coverslips were mounted using ProLong Gold antifade reagent with 4,6-diamidino-2-phenylindole (DAPI; Invitrogen). Immunofluorescence signal was detected using a LSM510 confocal microscope (Zeiss), and images were captured and analyzed using the Zeiss LSM Image Browser. For immunoblot analysis, human total PBMC or BHK cells were lysed with 100 μl of M-PER mammalian protein extraction reagent (Pierce) containing Halt protease inhibitor, EDTA and phosphatase inhibitor (Pierce). Equal amounts of protein were subjected to SDS-PAGE and transferred onto PVDF membranes. The blot was detected with anti-eIF2α and anti-phospho-eIF2α (Cell Signaling 9722) and developed with horseradish peroxidase-conjugated secondary antibody (Cell Signaling, 3597). Signals were visualized using SuperSignal West Pico chemiluminescent substrate (Pierce). C3a in plasma was measured by ELISA (Quidel A015).

### D. References

Alizadeh, A. A. et al. Distinct types of diffuse large B-cell lymphoma identified by gene expression profiling. Nature 403, 503-511 (2000).
Arimoto, K., Konishi, H. & Shimotohno, K. UbcH8 regulates ubiquitin and ISG15 conjugation to RIG-I. Mol. Immunol. 45, 1078-1084 (2008).
Atrasheuskaya, A. V., Fredeking, T. M. & Ignatyev, G. M. Changes in immune parameters and their correction in human cases of tick-borne encephalitis. Clin. Exp. Immunol. 131, 148-154 (2003).
Barba-Spaeth, G., Longman, R. S., Albert, M. L. & Rice, C. M. Live attenuated yellow fever 17D infects human DCs and allows for presentation of endogenous and recombinant T cell epitopes. J. Exp. Med. 202, 1179-1184 (2005).
Berlanga, J. J. et al. Antiviral effect of the mammalian translation initiation factor 2alpha kinase GCN2 against RNA viruses. EMBO J. 25, 1730-1740 (2006).
Brooks, J. P. & Lee, E. K. Analysis of the consistency of a mixed integer programming-based multi-category constrained discriminant model. Ann. Oper. Res. 164, 1-20 (2008).
Cancro, M. P. The BLyS/BAFF family of ligands and receptors: key targets in the therapy and understanding of autoimmunity. Ann. Rheum. Dis. 65 (suppl. 3), iii34-iii36 (2006).
Chen, J. P. et al. Dengue virus induces expression of CXC chemokine ligand 10/IFN-gamma-inducible protein 10, which competitively inhibits viral binding to cell surface heparan sulfate. J. Immunol. 177, 3185-3192 (2006).
Dabney, A. R. Classification of microarrays to nearest centroids. Bioinformatics 21, 4148-4154 (2005).
Diebold, S. S., Kaisho, T., Hemmi, H., Akira, S. & Reis e Sousa, C. Innate antiviral responses by means of TLR7-mediated recognition of single-stranded RNA. Science 303, 1529-1531 (2004).
Frauwirth, K. A. et al. The CD28 signaling pathway regulates glucose metabolism. Immunity 16, 769-777 (2002).
Gallagher, R. J., Lee, E. K. & Patterson, D. A. Constrained discriminant analysis via 0/1 mixed integer programming. Ann. Oper. Res. 74, 65-88 (1997).
Kaufman, R. J. Stress signaling from the lumen of the endoplasmic reticulum: coordination of gene transcriptional and translational controls. Genes Dev. 13, 1211-1233 (1999).
Kedersha, N. & Anderson, P. Mammalian stress granules and processing bodies. Methods Enzymol. 431, 61-81 (2007).
Lee, E. K. Large-scale optimization-based classification models in medicine and biology. Ann. Biomed. Eng. 35, 1095-1109 (2007).
Mehlhop, E. & Diamond, M. S. Protective immune responses against West Nile virus are primed by distinct complement activation pathways. J. Exp. Med. 203, 1371-1381 (2006).
Miller, J. D. et al. Human effector and memory CD8+ T cell responses to smallpox and yellow fever vaccines. Immunity 28, 710-722 (2008).
Monath, T. P. Yellow fever vaccine. Expert Rev. Vaccines 4, 553-574 (2005).
Monath, T. P., Cetron, M. & Teuwen, D. E. Yellow fever vaccine. in Vaccines 5th ed. (Saunders Elsevier, Philadelphia, 2008).
Pantaleo, G. HIV-1 T-cell vaccines: evaluating the next step. Lancet Infect. Dis. 8, 82-83 (2008).
Potti, A. et al. Genomic signatures to guide the use of chemotherapeutics. Nat. Med. 12, 1294-1300 (2006).
Querec, T. et al. Yellow fever vaccine YF-17D activates multiple dendritic cell subsets via TLR2, 7, 8, and 9 to stimulate polyvalent immunity. J. Exp. Med. 203, 413-424 (2006).
Ramilo, O. et al. Gene expression patterns in blood leukocytes discriminate patients with acute infections. Blood 109, 2066-2077 (2007).
Richter, J. D. & Sonenberg, N. Regulation of cap-dependent translation by eIF4E inhibitory proteins. Nature 433, 477-480 (2005).
Ron, D. & Walter, P. Signal integration in the endoplasmic reticulum unfolded protein response. Nat. Rev. Mol. Cell. Biol. 8, 519-529 (2007).
Rothenfusser, S. et al. The RNA helicase Lgp2 inhibits TLR-independent sensing of viral replication by retinoic acid-inducible gene-I. J. Immunol. 175, 5260-5268 (2005).
Shirato, K., Kimura, T., Mizutani, T., Kariwa, H. & Takashima, I. Different chemokine expression in lethal and non-lethal murine West Nile virus infection. J. Med. Virol. 74, 507-513 (2004).
Sorlie, T. et al. Gene expression patterns of breast carcinomas distinguish tumor subclasses with clinical implications. Proc. Natl. Acad. Sci. USA 98, 10869-10874 (2001).
Staub, O. Ubiquitylation and isgylation: overlapping enzymatic cascades do the job. Sci. STKE 2004, pe43 (2004).
Steinman, R. M. & Banchereau, J. Taking dendritic cells into medicine. Nature 449, 419-426 (2007).
Takeuchi, O. & Akira, S. Recognition of viruses by innate immunity Immunol. Rev. 220, 214-224 (2007).
Theiler, M. & Smith, H. H. The use of yellow fever virus modified by in vitro cultivation for human immunization. J. Exp. Med. 65, 787-800 (1937); Rev. Med. Virol. 10, 6-16; discussion 13-15 (2000).
Wong, J. J., Pung, Y. F., Sze, N. S. & Chin, K. C. HERC5 is an IFN-induced HECT-type E3 protein ligase that mediates type I IFN-induced ISGylation of protein targets. Proc. Natl. Acad. Sci. USA 103, 10735-10740 (2006).
Woodland, R. T., Schmidt, M. R. & Thompson, C. B. BLyS and B cell homeostasis. Semin. Immunol. 18, 318-326 (2006).
Zhao, F. Q. & Keating, A. F. Functional properties and genomics of glucose transporters. Curr. Genomics 8, 113-128 (2007).

What is claimed is:

1. A method for accessing the efficacy of a vaccine comprising identifying a differential expression signature of a tissue sample from an immunized subject, wherein the presence or absence of one or more innate response elements in expression signature indicates the presence of an adaptive immune response, and wherein the presence of an adaptive immune response indicates an efficacious vaccine.

2. The method of claim 1, wherein the vaccine is a live attenuated vaccine.

3. The method of claim 1, wherein the vaccine is a killed vaccine.

4. The method of claim 1, wherein the vaccine is a subunit vaccine.

5. The method of claim 1, wherein the vaccine is directed against a virus.

6. The method of claim 5, wherein the virus is a DNA virus.

7. The method of claim 6, wherein the virus is a double stranded DNA (dsDNA) virus.

8. The method of claim 7, wherein the dsDNA virus selected from the virus families consisting of Herpesviridae, Adenoviridae, Pappilomaviridae, and Poxyiridae.

9. The method of claim 6, wherein the virus is a single stranded DNA (ssDNA) virus.

10. The method of claim 5, wherein the virus is an RNA virus.

11. The method of claim 10, wherein the RNA virus is a double stranded RNA (dsRNA) virus.

12. The method of claim 11, wherein the dsRNA virus is from the family Reoviridae.

13. The method of claim 10, wherein the RNA virus is a positive-sense single stranded RNA (ssRNA) virus.

14. The method of claim 13, wherein the positive sense ssRNA virus is selected from the viral families consisting of Coronaviridae, Flaviviridae, Picornaviridae, and Togaviridae.

15. The method of claim 14, wherein the virus is Yellow Fever.

16. The method of claim 10, wherein the RNA virus is a negative-sense ssRNA virus.

17. The method of claim 16, wherein the negative sense ssRNA virus is selected from the viral families consisting of Filoviridae, Paramyxoviridae, Orthomyxoviridae, Rhabdoviridae, and Bunyaviridae.

18. The method of claim 1, wherein the vaccine is directed against a bacteria.

19. The method of claim 18, wherein the bacteria is a gram positive bacteria.

20. The method of claim 18, wherein the bacteria is a gram negative bacteria.

21. The method of claim 1, wherein the vaccine is directed against a fungi.

22. The method of claim 1, wherein the vaccine is directed against a parasite.

23. The method of claim 1, wherein the vaccine is directed against a cancer.

24. The method of claim 1, wherein the innate response element is an innate sensing receptor.

25. The method of claim 1, wherein the innate response element is a cytoplasmic receptor for oligodenylate synthetases.

26. The method of claim 1, wherein the innate response element is a transcription factors that regulate type I interferon expression.

27. The method of claim 1, wherein the innate response element is a gene in the complement pathway.

28. The method of claim 27, wherein the innate response element is C1QB.

29. The method of claim 1, wherein the innate response element regulates glucose transport and glycolysis.

30. The method of claim 29, wherein the innate response element is SLC2A6.

31. The method of claim 1, wherein the innate response element regulates protein synthesis in response to stress.

32. The method of claim 31, wherein the innate response element is EIF2AK4.

33. The method of claim 1, wherein the innate response element is a TNF receptor.

34. The method of claim 33, wherein the innate response element is TNF receptor superfamily receptor 17 (TNFRSF17).

35. The method of claim 1, wherein the adaptive immune response is a T cell response.

36. The method of claim 1, wherein the adaptive immune response is an antibody response.

37. The method of claim 1, wherein the differential expression signature is created by measuring the differential expression profile of a tissue sample from an immunized subject and identifying innate response elements with significant expression through computational analysis, wherein the innate response elements with significant expression comprise the expression signature of the sample.

38. The method of claim 37, wherein the computational analysis is discriminant analysis via mixed integer programming (DAMIP).

39. the method of claim 37, wherein the differential expression profile is created by measuring expression via western blot, RT-PCR, protein array, or gene array.

40. A method for measuring the efficacy of a vaccine comprising identifying a differential expression signature of a tissue sample from an immunized subject, wherein the presence or absence of one or more innate response elements in expression signature indicates the presence of an adaptive immune response, wherein the level of expression of the innate response elements correlates with the strength of the adaptive immune response, and wherein strength of the adaptive immune response indicates the level of efficacy of the vaccine.

41. A method for identifying a differential expression signature of an innate immune response element comprising comparing the expression profile of one or more innate response elements in a tissue sample of an immunized subject to a control sample, wherein innate response elements with significant differential expression are then correlated with an adaptive immune response using computational analysis; and wherein the innate response elements displaying correlation to an adaptive immune response comprise the differential expression signature.

42. The method of claim 41, wherein the computational analysis is discriminant analysis via mixed integer programming (DAMIP).

43. A system for determining a differential expression signature comprising a computer, a differential expression array, and software which takes the measurements from the array and applies the expression profile results to a computational analysis algorithm.

44. The method of claim 43, wherein the computational analysis algorithm is discriminant analysis via mixed integer programming (DAMIP).

45. A vaccine comprising one or more immunogenic elements which stimulate an adaptive immune response and one or more regulatory elements to stimulate or inhibit expression of one or more innate response elements.

46. A method of increasing the efficacy of a vaccine comprising modifying the vaccine to stimulate or inhibit one or more innate response elements.
